(19) United States
(12) Patent Application Publication
(10) Pub. No.: US 2026/0043008 A1
(43) Pub. Date: Feb. 12, 2026

(54) CELL CRYOPRESERVATIVE FORMULATIONS AND METHODS OF USE

(71) Applicant: ASTELLAS INSTITUTE FOR REGENERATIVE MEDICINE, Westborough, MA (US)

(72) Inventors: Samantha Jean PAULSEN, Westborough, MA (US); Hiroyuki KOJIMA, Shizuoka-Ken (JP); Shohei HACHIYA, Ibaraki (JP); Atushi INOUE, Ibaraki (JP)

(21) Appl. No.: 19/104,022

(22) PCT Filed: Aug. 22, 2023
(86) PCT No.: PCT/US2023/072683

§ 371 Date: Feb. 14, 2025

Related U.S. Application Data
(60) Provisional application No. 63/400,382, filed on Aug. 23, 2022.
(60) Provisional application No. 63/430,976, filed on Dec. 7, 2022.

(51) Int. Cl. C12N 5/00; A61K 35/30

(52) U.S. Cl. CPC C12N 5/525; A61K 35/30

(57) ABSTRACT

Some embodiments relate to improved cryopreservative formulations and improved methods for cryopreserving and thawing cells. These formulations facilitate high density, low volume cell cryopreservation strategies which overcome the need for washing of cells post-thaw, and therefore result in greater cell recovery and viability. Pharmaceutical compositions comprising a cell population and a cryopreservative formulation as provided herein are also provided. Methods for treating a subject having a disease or condition that benefits from transplantation of a cryopreserved and thawed cell population are also provided.

### RELATED APPLICATIONS

This application is a national phase filing under 35 C.F.R. § 371 of and claims priority to PCT Patent Application No. PCT/US2023/072683, filed on Aug. 22, 2023, which claims the benefit under 35 U.S.C. § 119(e) of U.S. Provisional Application Ser. No. 63/430,976, filed Dec. 7, 2022, entitled “CELL CRYOPRESERVATIVE FORMULATIONS AND METHODS OF USE” and U.S. Provisional Application Ser. No. 63/400,382, filed Aug. 23, 2022, entitled “CELL CRYOPRESERVATIVE FORMULATIONS AND METHODS OF USE”, the entire contents of each of which are incorporated herein by reference.

### BACKGROUND

Effective cell cryopreservation is an important aspect of many cell therapy strategies. The ability to freeze, store for various periods of time, and optionally transport cell-based therapies in a frozen state, expands their clinical use. Improper cryopreservation can lead to cellular damage including changes in cell characteristics, function, metabolic activity, and cell death.

### SUMMARY

The ability to cryopreserve cell-based therapeutics decouples the manufacture and clinical use of cellular therapies in time and location. The design and selection of suitable cryoprotectant formulations is important to ensure cell survival and maintenance of function. This disclosure provides novel cryoprotective formulations that can be used to cryopreserve and optionally transport cell populations, including those to be used as therapeutics. Importantly, this disclosure contemplates using such formulations to cryopreserve cell populations, at high density and in low volume, so that subsequent thawing is achieved by addition of a diluent, followed by administration to a subject, without the need to wash the cells post-thaw. In this way, the methods allow for freezing and thawing of cell-based therapeutics with minimal processing and manipulation. This reduces the likelihood of cell loss (e.g., cell death) that can result from manipulation of cells in the post-thaw period.

This disclosure therefore provides cryoprotective formulations and methods of use thereof. The cryoprotective formulations may be used with a variety of cell types. The cryoprotective formulations provided herein result in greater post-thaw viability and cell growth when compared to prior art methods for freezing cells and commercially available cryoprotective formulations. While ophthalmologic applications are exemplified herein, other applications are also contemplated and embraced by the present disclosure.

Some aspects of the present disclosure provide formulations comprising about 4-10% (v/v) cryoprotectant, about 2-8% (w/v) albumin, about 0-1.5% (w/v) glucose, and a buffer.

In some embodiments, the cryoprotectant is selected from DMSO, glycerol, and ethylene glycol.

In some embodiments, the cryoprotectant is DMSO.

In some embodiments, the formulation comprises about 4-6% (v/v) cryoprotectant.

In some embodiments, the formulation comprises about 0.08-0.10% (w/v) glucose.

In some embodiments, the formulation comprises about 5% (v/v) DMSO, about 2.5% (w/v) albumin, about 0.09% (w/v) glucose, and a buffer.

In some embodiments, the formulation comprises about 0.6% (w/v) glucose.

In some embodiments, the formulation comprises about 5% DMSO, about 2.5% albumin, about 0.6% glucose, and a buffer.

In some embodiments, albumin is human albumin.

In some embodiments, albumin is recombinant human albumin.

In some embodiments, the buffer is buffered saline.

In some embodiments, the buffer is phosphate-buffered saline (PBS).

In some embodiments, the buffered saline comprises Ca2+ and Mg2+.

In some embodiments, the buffered saline does not comprise Ca2+ and Mg2+.

In some embodiments, the formulation further comprises a cell population or a tissue.

In some embodiments, the cell population is a dissociated cell population.

In some embodiments, the cell population comprises a cell monolayer, a cell sheet, cellular clusters, cellular spheres or cellular aggregates.

In some embodiments, the cell population comprises retinal pigment epithelium (RPE) cells, photoreceptor rescue cells, photoreceptor progenitor cells, or corneal endothelial cells.

In some embodiments, the formulation is cryopreserved.

In some embodiments, the formulation does not comprise a polymeric excipient.

In some embodiments, the formulation does not comprise dextran.

Some aspects of the present disclosure provide cell preparations comprising (a) the formulation described herein and (b) a population of cells.

In some embodiments, the population of cells comprises retinal pigment epithelium (RPE) cells, photoreceptor rescue cells, photoreceptor progenitor cells or corneal endothelial cells.

In some embodiments, the population of cells is at a concentration of about 300 cells-1000 cells/μL in the cell preparation. In some embodiments, the population of cells is at a concentration of about 300 cells-10,000 cells/μL in the cell preparation. In some embodiments, the population of cells is at a concentration of about 1000 cells-10,000 cells/μL in the cell preparation.

In some embodiments, the population of cells is at a concentration of about 3,000 cells-5,000 cells/μL or at a concentration of about 3,000 cells-4,000 cells/μL in the cell preparation.

In some embodiments, the population of cells is at a concentration of about 10,000 cells-100,000 cells/μL in the cell preparation.

In some embodiments, the population of cells is at a concentration of about 15,000 cells-150,000 cells/μL in the cell preparation.

In some embodiments, the population of cells is at a concentration of about 15,000-50,000 cells/μL in the cell preparation.

In some embodiments, the cell preparation described herein is in a cryopreservation vial, syringe, ampoule, bottle or cooler package.

In some embodiments, about 10 to about 500 μL of the preparation are in the vial, syringe, ampoule, bottle or cooler package.

In some embodiments, about 10⁵ to about 10⁷ cells are present in the vial, syringe, ampoule, bottle or cooler package. In some embodiments, about 4×10⁵ cells to about 5×10⁵ cells are present in the vial. In some embodiments, about 4.5×10⁵ cells are present in the vial. In some embodiments, about 1×10⁶ cells to about 2×10⁶ cells are present in the vial. In some embodiments, about 1.5×10⁶ cells are present in the vial. In these embodiments, the cells are RPE cells. Similar cell numbers may be present in an syringe, ampoule, bottle or cooler package.

In some embodiments, about 0.5×10⁶ cells to about 1.5×10⁶ cells are present in the vial. In some embodiments, about 1×10⁶ cells are present in the vial. In some embodiments, about 5×10⁶ cells to about 7×10⁶ cells are present in the vial. In some embodiments, about 6×10⁶ cells are present in the vial. In these embodiments, the cells are PRC cells. Similar cell numbers may be present in an syringe, ampoule, bottle or cooler package.

In some embodiments, about 10⁵ to 5×10⁶ cells are present in the vial, syringe, ampoule, bottle or cooler package.

In some embodiments, about 10⁵ to 2×10⁶ cells are present in the vial, syringe, ampoule, bottle or cooler package.

In some embodiments, the cell preparation is cryopreserved.

Some aspects of the present disclosure provide methods of preparing a cell preparation, comprising contacting a formulation described herein with a population of cells.

In some embodiments, the method described herein further comprising cryopreserving the cell preparation.

In some embodiments, the population of cells comprises retinal pigment epithelium (RPE) cells, photoreceptor rescue cells, photoreceptor progenitor cells or corneal endothelial cells.

Some aspects of the present disclosure provide methods of treating a subject having a disorder or condition comprising, thawing the cell preparation described herein and administering the cell preparation to the subject.

Some aspects of the present disclosure provide methods of treating a subject having a disorder or condition comprising, thawing the cell preparation described herein, diluting the cell preparation with a diluent at a ratio in the range of from 1:2 to 1:20 including a ratio selected from the group consisting of 1:2, 1:3, 1:4 and 1:5, or a ratio of 1:10 to 1:15 including 1:10, 1:11, 1:12, 1:13, 1:14 and 1:15, and administering the diluted cell preparation to the subject.

In some embodiments, the disorder or condition is selected from the group consisting of retinal detachment, retinal dysplasia, Angioid streaks, Myopic Macular Degeneration, or retinal atrophy or associated with a number of vision-altering ailments that result in photoreceptor damage and blindness, such as, for example, choroideremia, diabetic retinopathy, macular degeneration (e.g., age related macular degeneration), retinitis pigmentosa, and Stargardt's Disease (fundus flavimaculatus).

In some embodiments, the diluent is GS2, GS2 Plus, BSS, BSS Plus®, a dextran and HSA solution, PBS, DMEM, MEM, or albumin.

Some aspects of the present disclosure provide methods of treating a subject having a disorder or condition comprising, thawing the cell preparation described herein, diluting the cell preparation with a diluent at a ratio in the range of 1:2 to 1:20 including a ratio selected from the group consisting of 1:15, 1:16, 1:17, 1:18 and 1:19, and administering the diluted cell preparation to the subject.

In some embodiments, the disorder or condition is selected from the group consisting of retinal detachment, retinal dysplasia, Angioid streaks, Myopic Macular Degeneration, or retinal atrophy or associated with a number of vision-altering ailments that result in photoreceptor damage and blindness, such as, for example, choroideremia, diabetic retinopathy, macular degeneration (e.g., age related macular degeneration), retinitis pigmentosa, and Stargardt's Disease (fundus flavimaculatus).

In some embodiments, the diluent is GS2 or GS2 Plus, BSS, BSS Plus®, a dextran and HSA solution, PBS, DMEM, MEM, or albumin.

Some aspects of the present disclosure provide kits comprising (a) the cryopreserved cell preparation described herein, (b) a diluent, and (c) instructions for diluting the cell preparation prior to administration to a subject.

In some embodiments, the instructions further comprise instructions for thawing the cryopreserved cell preparation.

Some aspects of the present disclosure provide comprise the formulation described herein in combination with a population of cells.

In some embodiments, the population of cells comprises retinal pigment epithelium (RPE) cells, photoreceptor rescue cells, photoreceptor progenitor cells or corneal endothelial cells.

In some embodiments, the population of cells comprises neural cells, neural stem cells, or neural precursor cells.

Some aspects of the present disclosure provide pharmaceutical compositions comprising the formulation described herein, wherein the pharmaceutical composition is suitable for administration to a subject.

Some aspects of the present disclosure provide pharmaceutical compositions comprising the formulation described herein and a diluent, wherein the formulation to diluent ratio is in the range of 1:2 to 1:100, including 1:2 to 1:20, including 1:2 to 1:5, including 1:2, 1:3, 1:4, 1:5, or a ratio of 1:10 to 1:15 including 1:10, 1:11, 1:12, 1:13, 1:14 and 1:15, wherein the pharmaceutical composition is suitable for administration to a subject.

In some embodiments, the pharmaceutical composition is suitable for administration to the eye.

In some embodiments, the pharmaceutical composition is suitable for administration to the subretina.

Some aspects of the present disclosure provide pharmaceutical compositions comprising the cell preparation described herein, wherein the pharmaceutical composition is suitable for administration to a subject.

Some aspects of the present disclosure provide pharmaceutical compositions comprising the cell preparation described herein and a diluent, wherein the cell preparation to diluent ratio is in the range of 1:2 to 1:100, including 1:2 to 1:20, including 1:2 to 1:5, including 1:2, 1:3, 1:4, 1:5, or a ratio of 1:10 to 1:15 including 1:10, 1:11, 1:12, 1:13, 1:14 and 1:15, wherein the pharmaceutical composition is suitable for administration to a subject.

In some embodiments, the pharmaceutical composition is suitable for administration to the eye.

In some embodiments, the pharmaceutical composition is suitable for administration to the subretina.

Some aspects of the present disclosure provide cell preparations comprising the formulation described herein, wherein the formulation comprises a cell population, wherein the cell preparation is suitable for administration to a subject.

Some aspects of the present disclosure provide cell preparations comprising the formulation described herein and a diluent, wherein the formulation to diluent ratio is in the range of 1:2 to 1:100, including 1:2 to 1:20, including 1:2 to 1:5, including 1:2, 1:3, 1:4, 1:5, or a ratio of 1:10 to 1:15 including 1:10, 1:11, 1:12, 1:13, 1:14 and 1:15, wherein the formulation comprises a cell population, wherein the cell preparation is suitable for administration to a subject.

In some embodiments, the cell preparation is suitable for administration to the eye.

In some embodiments, the cell preparation is suitable for administration to the subretina.

In some embodiments, the pharmaceutical composition or the cell preparation is not washed prior to or after dilution with the diluent.

In some embodiments, the pharmaceutical composition or the cell preparation is not centrifuged prior to or after dilution with the diluent.

In some embodiments, the pharmaceutical composition or the cell preparation is not diluted (e.g., not diluted prior to administration to a subject).

In some embodiments, the formulation or the cell preparation is cryopreserved prior to dilution with the diluent.

In some embodiments, DMSO is present in detectable amounts at a level that is equal to or less than about 1% (v/v).

In some embodiments, DMSO is present in detectable amounts at a level that is equal to or less than about 0.037 mg/dose.

Some aspects of the present disclosure provide cell preparations comprising the formulation described herein and a population of cells, wherein the cell preparation is thawed by dilution with a diluent and is unwashed.

In some embodiments, the cell preparation is diluted with a diluent at a ratio selected from the group consisting of 1:2 to 1:100, including 1:2 to 1:20, including 1:2 to 1:5, including 1:2, 1:3, 1:4, 1:5, or at a ratio of 1:10 to 1:15 including 1:10, 1:11, 1:12, 1:13, 1:14 and 1:15.

In some embodiments, DMSO is present in detectable amounts at a level that is equal to or less than about 0.037 mg/dose.

In some embodiments, the diluent is GS2 or GS2 Plus.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

FIG. 1 shows pre-freeze cell viability of retinal pigment epithelial (RPE) cells prepared in formulations containing different concentrations of dimethyl sulfoxide (DMSO), albumin and glucose, as shown in Table 1. Cryopreservative formulation #1 and Cryopreservative formulation #1 (2^{nd}) refer to different preparations of the same formulation.

FIG. 2 shows post-thaw cell viability of RPE cells prepared in formulations containing different concentrations of DMSO, albumin and glucose, as shown in Table 1. The first bar of each doublet represents a manual (Trypan Blue) viability measurement, and the second bar of each doublet represents an automated viability measurement using the NC-200 device.

FIG. 3 shows post-thaw cell growth of RPE cells prepared in formulations containing different concentrations of DMSO, albumin and glucose, as shown in Table 1, evaluated using the CyQUANT® Cell Proliferation Assay. The first bar of each doublet represents a measurement performed at day 2 post-thaw, and the second bar of each doublet represents a measurement performed at day 4 post-thaw.

FIG. 4A is an image of cryopreserved RPE cells, including multinucleated cells, under a microscope, after being thawed, seeded and cultured in growth medium for 3 days. The arrows indicate multinucleated cells. FIG. 4B is a graph showing the quantitative analysis of multinucleated cells present in a post-thaw culture in Formulations A-E. The first bar of each grouping represents bi-nucleated cells, the second bar represents tri-nucleated cells, the third bar represents tetra-nucleated cells, and the fourth bar represents cells with five or more nuclei.

FIG. 5A shows post-thaw cell viability (circles above the bar graph), and post-thaw cell growth at day 2 (first bar of each doublet) and day 3 (second bar of each doublet) post-thaw, as assessed by DNA concentration, of RPE cells prepared in formulations containing 2.5% or 5% Glycerol (“G”), 7.5% or 10% Dextran 40 (“D”) and 2.5% rHA in DPBS (with or without Ca and Mg, denoted “PBS−” and “PBS+” respectively), as shown in Table 3. FIG. 5B is a graph showing the quantitative analysis of multinucleated cells present in a post-thaw culture in the formulations. The first bar of each grouping represents bi-nucleated cells, the second bar represents tri-nucleated cells, the third bar represents tetra-nucleated cells, and the fourth bar represents cells with five or more nuclei.

FIG. 6A shows post-thaw cell viability (circles above the bar graph), and post-thaw cell growth at day 2 (first bar of each doublet) and day 3 (second bar of each doublet) as assessed by DNA concentration, of RPE cells prepared in formulations containing 5% DMSO (D5) or 5% Glycerol +7.5% Dextran 40 (G5) or 7.5% Dextran 40 (D0) or 2.5% Glycerol+7.5% Dextran 40 (G2.5) or 2.5% Propanediol (P2.5)+7.5% Dextran 40 (P2.5), each formulation also containing 0.09% Glucose, 2.5% rHA in PBS (+Ca, +Mg, as shown in Table 4. FIG. 6B is a graph showing the quantitative analysis of multinucleated cells present in a post-thaw culture in the formulations. The first bar of each grouping represents bi-nucleated cells, the second bar represents tri-nucleated cells, the third bar represents tetra-nucleated cells, and the fourth bar represents cells with five or more nuclei.

FIG. 7A shows post-thaw cell viability of photoreceptor rescue cells (PRC) cells prepared in formulations containing 2.5% rHA, DPBS with Ca and Mg, 0.6% glucose, and different concentrations of DMSO (5% DMSO or 10% DMSO), and of PRC cells formulated in a commercially available CryoStor® CS10 cell freezing formulation which contains 10% DMSO, for comparison. FIG. 7B shows post-thaw cell viability of photoreceptor rescue cells (PRC) cells prepared in formulations containing 2.5% rHA, DPBS with Ca and Mg, 0.6% glucose, and different concentrations of DMSO (5% DMSO, 7.5% DMSO or 10% DMSO).

FIG. 8 shows post-thaw cell viability of corneal endothelial cells (CEC) cells prepared in formulations containing 2.5% rHA, 0.09% Glucose, DPBS with Ca and Mg and different concentrations of DMSO (5% or 10%) at a density of 30,000 cells/μL and a volume of 50 μL, as compared with post-thaw cell viability of CEC cells cryopreserved at lower cell density (3 million cells/mL) and in standard volume (1 mL/vial) in 2.5% rHA, 0.09% glucose, DPBS with Ca and Mg, and 5% DMSO concentration.

### DETAILED DESCRIPTION

Cell-based therapies including those involving transplantation of cells, e.g., in the context of a regenerative medicine approach, often require formulating, storing, transporting, and/or injecting delicate or fragile cells that can be damaged or lose repopulation capacity upon inappropriate handling, extensive manipulation and/or processing steps or exposure to non-physiological conditions.

The present disclosure provides formulations and methods for cell cryopreservation, cell storage, cell transport, and ultimately cell administration to a subject with minimal processing and manipulation of the cells, and increased viability post-thaw. The formulations provided herein have several advantages over currently available formulations. For example, in contrast to currently available cryopreservative formulations, the formulations provided herein require minimal post-thaw processing, and can be administered to a subject along with the therapeutic with little if any adverse effect such as toxicity. The formulations provided herein allow for cryopreservation at a high density in a low volume which offers a number of advantages including a thawing regimen that requires only a single step of adding a diluent. Since there is no need to wash the formulation away post-thaw, there is less chance of cell loss and cell damage, thereby increasing the therapeutic efficacy of the cryopreserved cellular preparation. Currently available cryoprotectants and cryoprotective formulations require extensive cell processing procedures that may be costly, time-consuming, prone to human error, and ultimately may diminish cell survival, re-plating efficiency, and repopulation capacity of the cells contained therein. Also, notably, increased viability has been observed for cell populations that are cryopreserved using the formulations provided herein, compared to prior art cryopreservative formulations and methods. Improved cell growth post-thaw has also been observed when using the formulations provided herein. The formulations provided herein may provide for improved functionality, survival and stability of cell populations.

In addition to the afore-mentioned advantages, the cryopreservative formulations of this disclosure are also surprisingly and unexpectedly associated with reduced frequency (or prevalence) of multinucleated cells in culture, particularly in RPE cell cultures. Multinucleated cells, such as multinucleated RPE cells, have been observed using other formulations. However, surprisingly, the cryopreservative formulations provided herein appear to reduce the frequency of such cells.

This disclosure provides novel cryoprotective formulations that minimally include a cryoprotectant and albumin. In certain embodiments the cryoprotective formulations minimally include a cryoprotectant, albumin and optionally a sugar. The cryoprotectant may be DMSO, glycerol, ethylene glycol, trehalose, or taurine. In some embodiments, the cryoprotectant is DMSO. The albumin may be human albumin. The sugar may be glucose. The formulation may further comprise a buffer or a buffered saline such as but not limited to phosphate-buffered saline (PBS). The formulation may have a pH that is about a physiological pH (e.g., in the range of 6-8). The cryoprotectant may be present in a range of about 1% to about 10% volume/volume (v/v). The cryoprotectant may be present in a range of about 2% to about 10% volume/volume (v/v). The cryoprotectant may be present in a range of about 3% to about 10% volume/volume (v/v). The cryoprotectant may be present in a range of about 4% to about 10% volume/volume (v/v), for example 4%, 5%, 6%, 7%, 8%, 9% or 10%. The albumin may be present in a range of about 2% to about 3% (w/v). The albumin may be present in a range of about 2% to about 10% (w/v). The albumin may be present in a range of about 2% to about 8% (w/v). The albumin may be present in a range of about 2% to about 7.5% (w/v). The albumin may be present in a range of about 3.5% to about 7.5% (w/v). The albumin may be present in a range of about 4% to about 7.5% (w/v). The albumin may be present in a range of about 4% to about 8% (w/v). The albumin may be present in a range of about 4% to about 8.5% (w/v). The albumin may be present in a range of about 4% to about 9% (w/v). The albumin may be present in a range of about 4% to about 10% (w/v). The sugar may be present in a range of about 0-about 1.5% (w/v).

In some embodiments, the cryopreservative formulation comprises about 4% to about 10% DMSO (v/v), about 2% to about 3% albumin (w/v), about 0-1.5% glucose (w/v) and buffer or buffered saline. The formulation may consist essentially of about 4% to about 10% DMSO (v/v), about 2% to about 3% albumin (w/v), about 0-1.5% glucose (w/v) and buffer or buffered saline. The formulation may consist of about 4% to about 10% DMSO (v/v), about 2% to about 3% albumin (w/v), about 0-1.5% glucose (w/v) and buffer or buffered saline. The albumin may be human albumin, including recombinant human albumin. The buffered saline may be phosphate buffered saline (PBS).

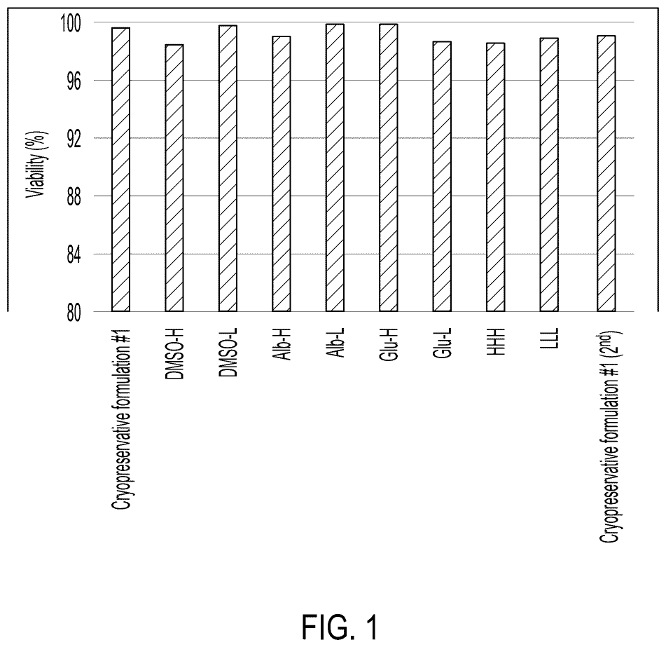

In some embodiments, the cryopreservative formulation comprises about 4% to about 6% DMSO (v/v), about 2% to about 3% albumin (w/v), about 0.08% to about 0.1% glucose (w/v) and buffer or buffered saline. In some embodiments, the cryopreservative formulation consists essentially of about 4% to about 6% DMSO (v/v), about 2% to about 3% albumin (w/v), about 0.08% to about 0.1% glucose (w/v) and buffer or buffered saline. In some embodiments, the cryopreservative formulation consists of about 4% to about 6% DMSO (v/v), about 2% to about 3% albumin (w/v), about 0.08% to about 0.1% glucose (w/v) and buffer or buffered saline. The albumin may be human albumin, including recombinant human albumin. The buffered saline may be phosphate buffered saline (PBS). Table 1 and FIG. 1 illustrate that RPE cells placed in formulations comprising about 4% to about 6% DMSO (v/v), about 2% to about 3% recombinant albumin (w/v), and about 0.08% to about 0.1% glucose (w/v) in PBS with calcium and magnesium had a viability exceeding 96%. When the cells were frozen in these formulations and then thawed, their viability ranged from 94-98%. Post-thaw cell growth was also relatively uniform across these formulations.

In some embodiments, the cryopreservative formulation comprises about 4% to about 10% DMSO (v/v), about 2% to about 7.5% albumin (w/v), about 0-1.5% glucose (w/v) and buffer or buffered saline. The formulation may consist essentially of about 4% to about 10% DMSO (v/v), about 2% to about 7.5% albumin (w/v), about 0-1.5% glucose (w/v) and buffer or buffered saline. The formulation may consist of about 4% to about 10% DMSO (v/v), about 2% to about 7.5% albumin (w/v), about 0-1.5% glucose (w/v) and buffer or buffered saline. The albumin may be human albumin, including recombinant human albumin. The buffered saline may be phosphate buffered saline (PBS).

In some embodiments, the cryopreservative formulation comprises about 4% to about 6% DMSO (v/v), about 2% to about 7.5% albumin (w/v), about 0.08% to about 0.1% glucose (w/v) and buffer or buffered saline. In some embodiments, the cryopreservative formulation consists essentially of about 4% to about 6% DMSO (v/v), about 2% to about 7.5% albumin (w/v), about 0.08% to about 0.1% glucose (w/v) and buffer or buffered saline. In some embodiments, the cryopreservative formulation consists of about 4% to about 6% DMSO (v/v), about 2% to about 7.5% albumin (w/v), about 0.08% to about 0.1% glucose (w/v) and buffer or buffered saline. The albumin may be human albumin, including recombinant human albumin. The buffered saline may be phosphate buffered saline (PBS). Table 1 and FIG. 1 illustrate that RPE cells placed in formulations comprising about 4% to about 6% DMSO (v/v), about 2% to about 3% recombinant albumin (w/v), and about 0.08% to about 0.1% glucose (w/v) in PBS with calcium and magnesium had a viability exceeding 96%. When the cells were frozen in these formulations and then thawed, their viability ranged from 94-98%. Post-thaw cell growth was also relatively uniform across these formulations.

Formulations comprising about 2.5% (v/v) DMSO, about 50 mM trehalose, about 7.5% (w/v) dextran-40, about 2.5% albumin (w/v) and buffered saline were associated with a reduced incidence of multinucleated RPE cells having more than 4 nuclei per cell, following freezing and thawing. DMSO at the 2.5% v/v level was more effective at reducing the incidence of multinucleated RPE cells having more than 4 nuclei per cell, as compared to DMSO at the 1% v/v level, following freezing and thawing. Formulations comprising even higher levels of DMSO (e.g., 5%) and optionally also containing ethylene glycol (e.g., 5%) were associated with reduced incidence of multinucleated cells having more than 3 nuclei per cell. Formulations comprising 5% glycerol did not give rise to cells having more than 4 nuclei per cell.

In some embodiments, the cryoprotectant is a cell-penetrating cryoprotectant such as DMSO, glycerol or ethylene glycol, all of which were found to be associated with multinucleation of cells of interest including but not limited to RPE cells, photoreceptor cells, photoreceptor progenitor cells, photoreceptor rescue cells, corneal endothelial cells, corneal endothelial progenitor cells, mesenchymal cells, mesenchymal stem cells, hematopoietic stem cells, neural or neuronal stem or progenitor cells, glial stem or progenitor cells, pancreatic stem or progenitor cells, beta cells, keratinocytes, chondrocytes, osteoblasts, osteoclasts, or cells within a population or tissue, e.g., a monolayer or sheet or aggregate or cluster of RPE cells, corneal endothelial cells, photoreceptor rescue cells, a pancreatic islet, or a skin graft.

In some embodiments, the cryoprotectant is a cell-penetrating cryoprotectant such as DMSO, glycerol or ethylene glycol, all of which were found to be associated with reduced incidence of multinucleated RPE cells.

Still other formulations comprise about 5% DMSO (v/v), about 2.5% albumin (w/v), about 0.09% glucose (w/v) and buffer or buffered saline. Other formulations comprise about 2.5% albumin (w/v), about 0.09% glucose (w/v), about 5% (v/v) glycerin, about 200 mM taurine, and buffer or buffered saline. Other formulation comprise 2.5% albumin (w/v), about 0.09% glucose (w/v), about 5% (v/v) glycerin, about 100 mM trehalose, and buffer or buffered saline.

Still other formulations comprise about 5% DMSO (v/v), about 2.5% albumin (w/v), about 0.6% glucose (w/v) and buffer or buffered saline. Other formulations comprise about 2.5% albumin (w/v), about 0.6% glucose (w/v), about 5% (v/v) glycerin, about 200 mM taurine, and buffer or buffered saline. Other formulation comprise 2.5% albumin (w/v), about 0.6% glucose (w/v), about 5% (v/v) glycerin, about 100 mM trehalose, and buffer or buffered saline.

Still other formulations comprise about 5% DMSO (v/v), about 7.5% albumin (w/v), about 0.6% glucose (w/v) and buffer or buffered saline. Other formulations comprise about 7.5% albumin (w/v), about 0.6% glucose (w/v), about 5% (v/v) glycerin, about 200 mM taurine, and buffer or buffered saline. Other formulation comprise 7.5% albumin (w/v), about 0.6% glucose (w/v), about 5% (v/v) glycerin, about 100 mM trehalose, and buffer or buffered saline.

The disclosure further contemplates that in any of the embodiments provided herein, “glycerol” could also encompass “glycerin” and conversely, in any of the embodiments provided herein, “glycerin” could also encompass “glycerol”.

The cryopreserved cell preparation may comprise, in some embodiments, the cells of interest (e.g., RPE cells, photoreceptor rescue cells, photoreceptor cells, corneal endothelial cells, corneal endothelial progenitors, neural cells or neural stem cells or neural progenitor cells or other cell types disclosed herein), 5% or 10% DMSO, 27 μg glucose, 750 μg recombinant human albumin, 110 μg sodium chloride, 26.6 μg sodium phosphate, 2.10 μg calcium chloride, 4.19 μg potassium chloride, 4.19 μg potassium phosphate monobasic, 2.10 μg magnesium chloride-6H₂O, 168 μg sodium chloride, and 24.1 μg sodium phosphate dibasic. The cryopreserved cell preparation may comprise, in some embodiments, the cells of interest (e.g., RPE cells), 5% or 10% DMSO. 0.9 μg/ml Glucose, 259.5 μg/ml Recombumin Elite (recombinant human albumin) and Dulbecco's phosphate buffered saline as a buffering agent.

Upon thaw and dilution in a diluent as provided herein, the final cell preparation to be administered to a subject may comprise, in some embodiments, the cells of interest (e.g., RPE cells), sodium hyaluronate, sodium chloride, potassium chloride, sodium phosphate dibasic dodecahydrate, dextrose (anhydrous), calcium chloride dihydrate, magnesium chloride hexahydrate, sodium acetate trihydrate, and sodium citrate dihydrate.

The cells may be cryopreserved at higher concentration (or density) compared to standard prior art techniques. For example, the cells may be cryopreserved at a concentration (or density) of about 3×10² cells per μL or higher, including about 10³ cells per μL, about 2×10³ cells per μL, about 3×10³ cells per μL, 10⁴ cells per μL or higher, including about 2×10⁴ cells per μL, about 3×10⁴ cells per μL, about 4×10⁴ cells per μL, about 5×10⁴ cells per μL, about 6×10⁴ cells per μL, about 7×10⁴ cells per μL, about 8×10⁴ cells per μL, about 9×10⁴ cells per μL, or about 10⁵ cells per μL, or higher. In some embodiments, the cell density is about 3×10² cells per μL. In some embodiments, the cell density is about 3.3×10³ cells per μL. In some embodiments, the cell density is about 5×10⁴ cells per μL.

In some embodiments, the cell density is in the range of about 10³ cells per μL to about 6×10⁴ cells per μL, including about 3×10³ cells per μL to about 5×10⁴ cells per μL. In some embodiments, the cell density is in the range of about 10⁴ cells per μL to about 6×10⁴ cells per μL, including 1.5×10⁴ cell per μL to about 5×10⁴ cells per μL, including about 2×10⁴ cells per μL to about 5×10⁴ cells per μL, and about 3×10⁴ cells per μL to about 5×10⁴ cells per μL. In some embodiments, the cell density is about 3×10² cells per μL. In some embodiments, the cell density is about 3.3×10³ cells per μL. In some embodiments, the cell density is about 5×10⁴ cells per μL.

In some embodiments, the number of cells per vial is about 10⁵ to 10⁷ cells per vial. In some embodiments, the number of cells per vial is about 450,000 to 1,500,000 cells per vial. In some embodiments, the number of cells per vial is about 1,750,000 to about 3,500,000 cells per vial.

Additionally, the volume of the cryopreserved cell preparation may be reduced compared to standard prior art techniques. For example, the cells may be cryopreserved in volumes ranging from about 10 μL to about 100 μL, in some embodiments, including volumes of about 10 μL to about 50 μL, including volumes of about 10 μL, about 20 μL, about 30 μL, about 40 μL, about 50 μL, about 60 μL, about 70 μL, about 80 μL, about 90 μL, or about 100 μL. In some embodiments, the cells may be cryopreserved in volumes ranging from about 10 μL to about 500 μL, in some embodiments, including volumes of 10 μL, about 20 μL, about 30 μL, about 40 μL, about 50 μL, about 60 μL, about 70 μL, about 80 μL, about 90 μL, about 100 μL, about 150 μL, about 200 μL, about 250 μL, about 300 μL, about 350 μL, about 400 μL, about 450 μL, or about 500 μL. Since the cells can be frozen in large numbers yet in low volumes, the frozen preparation can then be diluted with a suitable diluent to arrive at a desired volume and concentration, and then directly administered to a subject without the need for performing a washing step. Standard prior art techniques typically freeze lower concentrations of cells in larger volumes (e.g., on the order of 1 mL), and then require that the thawed preparation be washed, sometimes multiple times, in order to remove the cryoprotectant before administration to a subject.

In some embodiments, the cryoprotective formulation comprises, consists essentially of, or consists of about 4% to about 10% DMSO (w/v), about 2% to about 3% albumin (w/v), 0-1.5% glucose (w/v) and buffer or buffered saline, and is used to cryopreserve RPE cells. This formulation results in viability of the RPE cells, post thaw, of at least 90% or at least 91% or at least 92% or at least 93% or at least 94% or at least 95%. It also results in a lower frequency of multi-nucleated cells. In some embodiments, the cryoprotective formulation comprises, consists essentially of, or consists of about 10% DMSO (v/v), about 2.4% albumin (w/v), 0-1.5% glucose (w/v) and buffer.

In some embodiments, the cryoprotective formulation comprises, consists essentially of, or consists of about 4% to about 10% DMSO (v/v), about 2% to about 3% albumin (w/v), 0-1.5% glucose (w/v) and buffer, and is used to cryopreserve photoreceptor rescue cells, photoreceptor cells or photoreceptor progenitor cells. The DMSO may be used at about 5% or about 10%. The cells may be frozen at a concentration of about 50×10³ cells per μL. The formulation results in improved viability of the cells, post-thaw, compared to a commercially available cryoprotective formulation, CS10.

In some embodiments, the cryoprotective formulation comprises, consists essentially of, or consists of about 4% to about 10% DMSO (v/v), about 2% to about 3% albumin (w/v), 0-1.5% glucose (w/v) and buffer or buffered saline, and is used to cryopreserve corneal endothelial progenitor cells or corneal endothelial cells. The DMSO may be present at about 5% or about 10% (v/v). The cells may be frozen at a concentration of about 30×10³ cells per μL. The formulation results in improved viability of the cells, post-thaw, compared to the conventional cryoprotective formulation of 90% FBS (v/v) and 10% DMSO (v/v).

In some embodiments, the cryoprotective formulation comprises, consists essentially of, or consists of about 4% to about 10% DMSO (v/v), about 2% to about 3% albumin (w/v), 0-1.5% glucose (w/v) and buffer or buffered saline. The DMSO may be present at about 4% (v/v), about 5% (v/v), and about 6% (v/v). The albumin may be human albumin, including recombinant human albumin, and may be present at about 2% (w/v), or about 2.5% (w/v), or about 3% (w/v). The glucose may be absent, or it may be present at about 0.08% (w/v) or about 0.09% (w/v), or about 0.1% (w/v). The buffered saline may be phosphate buffered saline.

In some embodiments, the cryoprotective formulation comprises, consists essentially of, or consists of about 4% to about 6% DMSO (v/v), about 2% to about 3% albumin (w/v), 0-1.5% glucose (w/v) and buffer or buffered saline. The albumin may be present at about 2% (w/v), about 2.5% (w/v), or about 3% (w/v). The glucose may be present at about 0.08% (w/v), or about 0.09% (w/v), or about 0.10% (w/v).

In some embodiments, the cryoprotective formulation comprises, consists essentially of, or consists of about 4% to about 10% DMSO (v/v), about 2% to about 3% albumin (w/v), 0-1.5% glucose (w/v) and buffer or buffered saline. The DMSO may be present at about 4% (v/v), about 5% (v/v), and about 6% (v/v). The albumin may be human albumin, including recombinant human albumin, and may be present at about 2% (w/v), or about 2.5% (w/v), or about 3% (w/v), or about 4% (w/v), or about 5% (w/v), or about 6% (w/v), or about 7% (w/v), or about 7.5% (w/v), or about 8% (w/v), or about 9% (w/v) or about 10% w/v). The glucose may be absent, or it may be present at about 0.08% (w/v) or about 0.09% (w/v), or about 0.1% (w/v). The buffered saline may be phosphate buffered saline.

In some embodiments, the cryoprotective formulation comprises, consists essentially of, or consists of about 4% to about 6% DMSO (v/v), about 2% to about 3% albumin (w/v), 0-1.5% glucose (w/v) and buffer or buffered saline. The albumin may be present at about 2% (w/v), about 2.5% (w/v), or about 3% (w/v). In some embodiments, the cryoprotective formulation comprises, consists essentially of, or consists of about 4% to about 6% DMSO (v/v), about 2% to about 7.5% albumin (w/v), 0-1.5% glucose (w/v) and buffer or buffered saline. The albumin may be present at about 2% (w/v), about 5% (w/v), or about 7.5% (w/v). The glucose may be present at about 0.08% (w/v), or about 0.09% (w/v), or about 0.10% (w/v).

### Cryopreservative Formulations

In some embodiments, the formulations or preparations provided herein exhibit a physiological pH and a physiological osmotic pressure, also referred to as a physiological osmolarity. A physiological pH refers to a pH that is not cytotoxic and resembles the pH of the cell or tissue in its natural environment. For most cells and tissues, a physiological pH is a pH of about 6.8 to about 7.8, for example, a pH of about 7 to about 7.7, a pH of about 7.2 to about 7.6, a pH of about 7.2 to about 7.4, or a pH of about 7.4 to about 7.5. Accordingly, in some embodiments, the formulations or preparations provided herein exhibit a pH of about 7.0, about 7.1, about 7.2, about 7.3, about 7.4, about 7.5, about 7.6, about 7.7, or about 7.8. A physiological osmotic pressure refers to an osmotic pressure that is not cytotoxic and resembles the osmotic pressure of the cell or tissue in its natural environment. For some cells and tissues, a physiological osmotic pressure is about 270 to about 345 mOsm/l, for example, about 280 to about 330 mOsm/l, about 290 to about 325 mOsm/l, about 300 to about 315 mOsm/l. In some embodiments, a physiological osmotic pressure is about 300, about 305, about 310, about 315, about 320, or about 325 mOsm/l. In some embodiments, the formulations or preparations provided herein exhibit a physiological pH and an osmotic pressure that is greater than physiological osmotic pressure, for example, about 350 to about 450 mOsm/l, for example, about 360 to about 440 mOsm/l, about 370 to about 430 mOsm/l, about 380 to about 420 mOsm/l, or about 390 to about 410 mOsm/l. In some embodiments, an osmotic pressure that is greater than physiological osmotic pressure is about 350, about 355, about 360, about 365, about 370, about 375, about 380, about 385, about 390, about 395, about 400, about 405, about 410, about 415, about 420, about 425, about 430, about 435, about 440, about 445 or about 450 mOsm/1. In some embodiments, the formulations or preparations provided herein have an osmolality of about 1250 mOsm/kg, for example about 1250 mOsm/kg, about 1000 mOsm/kg, about 900 mOsm/kg, about 800 mOsm/kg, about 700 mOsm/kg, about 600 mOsm/kg, about 500 mOsm/kg, about 400 mOsm/kg or about 300 mOsm/kg. In some embodiments, the formulations or preparation provided herein have an osmolality about 300-450 mOsm/kg, about 300-400 mOsm/kg, about 300-450 mOsm/kg, about 350-450 mOsm/kg or about 350-400 mOsm/kg.

In some embodiments, the cryopreservative formulations provided herein comprise (a) a cryoprotectant; (b) albumin; (c) a buffer, maintaining the solution at a physiological pH; and (d) glucose.

In some embodiments, the formulations comprise a cryoprotectant. Cryoprotectants are used to protect cells from damage during the freezing process. In some embodiments, the formulations comprise a cryoprotectant selected from dimethyl sulfoxide (DMSO), glycerol, and ethylene glycol. In some embodiments, the cryoprotectant is DMSO. In some embodiments, the formulations comprise about 4%, about 4.5%, about 5%, about 5.5%, about 6%, about 6.5%, about 7%, about 7.5%, about 8%, about 8.5%, about 9%, about 9.5%, or about 10% (volume/volume, v/v) DMSO. In some embodiments, the solution comprises about 4%-10%, about 4%-9.5%, about 4%-9%, about 4%-8.5%, about 4%-8%, about 4%-7.5%, about 4%-7%, about 4%-6.5%, about 4%-6%, about 4%-5.5%, about 4%-5%, about 4%-4.5%, about 4.5%-10%, about 4.5%-9.5%, about 4.5%-9%, about 4.5%-8.5%, about 4.5%-8%, about 4.5%-7.5%, about 4.5%-7%, about 4.5%-6.5%, about 4.5%-6%, about 4.5%-5.5%, about 4.5%-5%, about 5%-10%, about 5%-9.5%, about 5%-9%, about 5%-8.5%, about 5%-8%, about 5%-7.5%, about 5%-7%, about 5%-6.5%, about 5%-6%, about 5%-5.5%, about 5.5%-10%, about 5.5%-9.5%, about 5.5%-9%, about 5.5%-8.5%, about 5.5%-8%, about 5.5%-7.5%, about 5.5%-7%, about 5.5%-6.5%, about 5.5%-6%, about 6%-10%, about 6%-9.5%, about 6%-9%, about 6%-8.5%, about 6%-8%, about 6%-7.5%, about 6%-7%, about 6%-6.5%, about 6.5%-10%, about 6.5%-9.5%, about 6.5%-9%, about 6.5%-8.5%, about 6.5%-8%, about 6.5%-7.5%, about 6.5%-7%, about 7%-10%, about 7%-9.5%, about 7%-9%, about 7%-8.5%, about 7%-8%, about 7%-7.5%, about 7.5%-10%, about 7.5%-9.5%, about 7.5%-9%, about 7.5%-8.5%, about 7.5%-8%, about 8%-10%, about 8%-9.5%, about 8%-9%, about 8%-8.5%, about 8.5%-10%, about 8.5%-9.5%, about 8.5%-9%, about 9%-10%, about 9%-9.5%, or about 9.5%-10% (v/v) DMSO. In some embodiments, the formulations comprise about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9% or about 10% (volume/volume, v/v) DMSO.

In some embodiments, the formulations comprise albumin. In some embodiments, the albumin is recombinant albumin. In some embodiments, the albumin is recombinant human (rh) albumin. The term “recombinant albumin” is interchangeably used with the term “rA” or “rAlbumin”. The term “recombinant human albumin” is interchangeably used with the term “rHA”, “rHSA”, “rhAlbumin”. In some embodiments, the cryopreservative formulations comprises about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0% about 5.1%, about 5.2%, about 5.3%, about 5.4%, about 5.5%, about 5.6%, about 5.7%, about 5.8%, about 5.9%, about 6.0%, about 6.1%, about 6.2%, about 6.3%, about 6.4%, about 6.5%, about 6.6%, about 6.7%, about 6.8%, about 6.9%, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.6%, about 8.7%, about 8.8%, about 8.9%, about 9.0%, about 9.1%, about 9.2%, about 9.3%, about 9.4%, about 9.5%, about 9.6%, about 9.7%, about 9.8%, about 9.9%, or about 10.0% (w/v) albumin.

In some embodiments, the cryopreservative formulations comprise about 2.0%-10.0%, about 2.0%-9.5%, about 2.0%-9.0%, about 2.0%-8.5%, about 2.0%-8.0%, about 2.0%-7.5%, about 2.0%-7.0%, about 2.0%-6.5%, about 2.0%-6.0%, about 2.0%-5.5%, about 2.0%-5.0%, about 2.0%-4.5%, about 2.0%-4.0%, about 2.0%-3.5%, about 2.0%-3.0%, about 2.0%-2.5%, about 2.5%-10.0%, about 2.5%-9.5%, about 2.5%-9.0%, about 2.5%-8.5%, about 2.5%-8.0%, about 2.5%-7.5%, about 2.5%-7.0%, about 2.5%-6.5%, about 2.5%-6.0%, about 2.5%-5.5%, about 2.5%-5.0%, about 2.5%-4.5%, about 2.5%-4.0%, about 2.5%-3.5%, about 2.5%-3.0%, about 3.0%-10.0%, about 3.0%-9.5%, about 3.0%-9.0%, about 3.0%-8.5%, about 3.0%-8.0%, about 3.0%-7.5%, about 3.0%-7.0%, about 3.0%-6.5%, about 3.0%-6.0%, about 3.0%-5.5%, about 3.0%-5.0%, about 3.0%-4.5%, about 3.0%-4.0%, about 3.0%-3.5%, about 3.5%-10.0%, about 3.5%-9.5%, about 3.5%-9.0%, about 3.5%-8.5%, about 3.5%-8.0%, about 3.5%-7.5%, about 3.5%-7.0%, about 3.5%-6.5%, about 3.5%-6.0%, about 3.5%-5.5%, about 3.5%-5.0%, about 3.5%-4.5%, about 3.5%-4.0%, about 4.0%-10.0%, about 4.0%-9.5%, about 4.0%-9.0%, about 4.0%-8.5%, about 4.0%-8.0%, about 4.0%-7.5%, about 4.0%-7.0%, about 4.0%-6.5%, about 4.0%-6.0%, about 4.0%-5.5%, about 4.0%-5.0%, about 4.0%-4.5%, about 4.5%-10.0%, about 4.5%-9.5%, about 4.5%-9.0%, about 4.5%-8.5%, about 4.5%-8.0%, about 4.5%-7.5%, about 4.5%-7.0%, about 4.5%-6.5%, about 4.5%-6.0%, about 4.5%-5.5%, about 4.5%-5.0%, about 5.0%-10.0%, about 5.0%-9.5%, about 5.0%-9.0%, about 5.0%-8.5%, about 5.0%-8.0%, about 5.0%-7.5%, about 5.0%-7.0%, about 5.0%-6.5%, about 5.0%-6.0%, about 5.0%-5.5%, about 5.5%-10.0%, about 5.5%-9.5%, about 5.5%-9.0%, about 5.5%-8.5%, about 5.5%-8.0%, about 5.5%-7.5%, about 5.5%-7.0%, about 5.5%-6.5%, about 5.5%-6.0%, about 6.0%-10.0%, about 6.0%-9.5%, about 6.0%-9.0%, about 6.0%-8.5%, about 6.0%-8.0%, about 6.0%-7.5%, about 6.0%-7.0%, about 6.0%-6.5%, about 6.5%-10.0%, about 6.5%-9.5%, about 6.5%-9.0%, about 6.5%-8.5%, about 6.5%-8.0%, about 6.5%-7.5%, about 6.5%-7.0%, about 7.0%-10.0%, about 7.0%-9.5%, about 7.0%-9.0%, about 7.0%-8.5%, about 7.0%-8.0%, about 7.0%-7.5%, about 8.0%-10.0%, about 8.0%-9.5%, about 8.0%-9.0%, about 8.0%-8.5%, about 8.5%-10.0%, about 8.5%-9.5%, about 8.5%-9.0%, about 9.0%-10.0%, or about 9.5%-10.0% (w/v) albumin.

In some embodiments, the cryopreservative formulations comprise about 2.0%-3.0%, about 2.0%-2.9%, about 2.0%-2.8%, about 2.0%-2.7%, about 2.0%-2.6%, about 2.0%-2.5%, about 2.0%-2.4%, about 2.0%-2.3%, about 2.0%-2.2%, about 2.0%-2.1%, about 2.1%-3.0%, about 2.1%-2.9%, about 2.1%-2.8%, about 2.1%-2.7%, about 2.1%-2.6%, about 2.1%-2.5%, about 2.1%-2.4%, about 2.1%-2.3%, about 2.1%-2.2%, about 2.2%-3.0%, about 2.2%-2.9%, about 2.2%-2.8%, about 2.2%-2.7%, about 2.2%-2.6%, about 2.2%-2.5%, about 2.2%-2.4%, about 2.2%-2.3%, about 2.3%-3.0%, about 2.3%-2.9%, about 2.3%-2.8%, about 2.3%-2.7%, about 2.3%-2.6%, about 2.3%-2.5%, about 2.3%-2.4%, about 2.4%-3.0%, about 2.4%-2.9%, about 2.4%-2.8%, about 2.4%-2.7%, about 2.4%-2.6%, about 2.4%-2.5%, about 2.5%-3.0%, about 2.5%-2.9%, about 2.5%-2.8%, about 2.5%-2.7%, about 2.5%-2.6%, about 2.6%-3.0%, about 2.6%-2.9%, about 2.6%-2.8%, about 2.6%-2.7%, about 2.7%-3.0%, about 2.7%-2.9%, about 2.7%-2.8%, about 2.8%-3.0%, about 2.8%-2.9%, or about 2.9%-3.0% (w/v) albumin.

In some embodiments, the cryopreservative formulations comprise a buffer. A buffer refers to an agent that can maintain the pH of a solution, preparation or formulation relatively stable by neutralizing added acid or base. Typically, a buffer comprises a weak conjugate acid-base pair, i.e., either a weak acid and its conjugate base, or a weak base and its conjugate acid. In some embodiments, the buffer comprised in the cryopreservative formulations provided herein is a water-based salt solution comprising disodium hydrogen phosphate (Na₂HPO₄) and sodium chloride (NaCl). In some embodiments, the buffer comprised in the formulations provided herein further comprises potassium chloride (KCl) and potassium dihydrogen phosphate (KH₂PO₄). In some embodiments, the formulations comprise a buffered saline such as phosphate-buffered saline (PBS). In some embodiments, the buffer is Dulbecco's phosphate-buffered saline (DPBS). In some embodiments, the buffer is 4-(2-hydroxyethyl)-1-piperazineethanesulfonic acid (HEPES). In some embodiments, the buffer is 3-(N-morpholino) propanesulfonic acid (MOPS). In some embodiments, the buffer is N,N-Bis-(2-hydroxyethyl)-2-aminoethanesulfonic acid (BES). In some embodiments, the buffer is 3-morpholino-2-hydroxypropanesulfonic acid sodium salt (MOPSO). In some embodiments, the buffer is N-tris(Hydroxymethyl) Methyl Glycine (Tricine). In some embodiments, the buffer is 2-[Tris(hydroxymethyl)methylamino]-1-ethanesulfonic acid (TES). In some embodiments, the buffer is 4-(2-Hydroxyethyl)-1-piperazinepropanesulfonic acid (EPPS or HEPPS).

In some embodiments, the buffer comprises divalent cations. Suitable divalent cations include, without limitation, e.g., Ca²⁺, Mg²⁺, Zn²⁺, Fe²⁺, Mn²⁺, Cr²⁺, Cu²⁺, Ba²⁺, and Sr²⁺. In some embodiments, the divalent cations comprise calcium. In some embodiments, the divalent cations comprise magnesium. In some embodiments, the divalent cations comprise a two or more different divalent cations, e.g., calcium and magnesium. In some embodiments, the buffer comprises calcium. In some embodiments, the buffer comprises a pharmaceutically acceptable calcium salt. In some embodiments, the buffer comprises magnesium. In some embodiments, the buffer comprises a pharmaceutically acceptable magnesium salt. In some embodiments of the solutions provided herein, the buffer comprises calcium chloride. In some embodiments, the buffer comprises calcium chloride dihydrate. In some embodiments, the buffer comprises magnesium chloride. In some embodiments, the buffer comprises magnesium chloride hexahydrate.

In some embodiments, the cryopreservative formulations provided herein comprise glucose. In some embodiments, the solution comprises about 0%-5.0% (w/v) glucose. For example, in some embodiments, the cryopreservative formulation comprises about 0%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 4%, about 4.1%, about 4.2%, about 4.3%, about 4.4%, about 4.5%, about 4.6%, about 4.7%, about 4.8%, about 4.9% or about 5% (w/v) glucose. The amount of glucose can be expressed as a range. The disclosure contemplates and herein provides any range of glucose selected from a lower limit of any one of the following and a upper limit of any one of the following. Any combination of these is disclosed herein. The lower limit of the range includes any one of the following 0%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 4%, 4.1%, 4.2%, 4.3%, 4.4%, 4.5%, 4.6%, 4.7%, 4.8%, and 4.9% and the range includes an upper limit of any of the following 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 4%, 4.1%, 4.2%, 4.3%, 4.4%, 4.5%, 4.6%, 4.7%, 4.8%, 4.9% or 5% (w/v) glucose. This disclosure contemplates any range of glucose having any combination of lower limit as disclosed and upper limit as disclosed. For example, provided herein are ranges such as about 0-. 5%, about 0-1%, about 0-1.5%, about 0-2%, about 0-2.5%, about 0-3%, about 0-3.5%, about 0-4%, about 0-4.5%, about 0-5%, about 0.5-1%, about 0.5-1.5%, about 0.5-2%, about 0.5-2.5%, about 0.5-3%, about 0.5-3.5%, about 0.5-4%, about 0.5-4.5%, about 0.5-5%, about 1-1.5%, about 1-2%, about 1-2.5%, about 1-3%, about 1-3.5%, about 1-4%, about 1-4.5%, about 1-5%, about 1.5-2%, about 1.5-2.5%, about 1.5-3%, about 1.5-3.5%, about 1.5-4%, about 1.5-4.5%, about 1.5-5%, about 2-2.5%, about 2-3%, about 2-3.5%, about 2-4%, about 2-4.5%, about 2-5%, about 2.5-3%, about 2.5-3.5%, about 2.5-4%, about 2.5-4.5%, about 2.5-5%, about 3-3.5%, about 3-4%, about 3-4.5%, about 3-5%, about 3.5-4%, about 3.5-4.5%, about 3.5-5%, about 4-4.5%, about 4-5%, and about 4.5-5% (w/v) glucose. In some embodiments, the solution comprises about 0-1.5% (w/v) glucose. It is to be understood that a range of about 1-1.5%, as an example, means about 1% to about 1.5%. A similar meaning is imparted to each and every range recited herein.

In some embodiments, the formulation comprises about 4-10% (v/v) cell cryoprotectant, about 2-3% (w/v) albumin, about 0-1.5% (w/v) glucose, and a buffer, optionally as a buffered saline. In some embodiments, the formulation comprises about 4-10% (v/v) cell cryoprotectant, about 2-3% (w/v) albumin, about 0.08-0.10% (w/v) glucose, and a buffer, optionally as a buffered saline. In some embodiments, the formulation comprises about 4-6% (v/v) cell cryoprotectant, about 2-3% (w/v) albumin, about 0.08-0.10% (w/v) glucose, and a buffer, optionally as a buffered saline.

In some embodiments, the formulation comprises about 4-10% (v/v) DMSO, about 2-3% (w/v) albumin, about 0-1.5% (w/v) glucose, and a buffer. In some embodiments, the formulation comprises about 4-10% (v/v) DMSO, about 2-3% (w/v) albumin, about 0.08-0.10% (w/v) glucose, and a buffer. In some embodiments, the formulation comprises about 4-6% (v/v) DMSO, about 2-3% (w/v) albumin, about 0.08-0.10% (w/v) glucose, and a buffer. In some embodiments, the formulation comprises about 5% (v/v) DMSO, about 2.5% (w/v) albumin, about 0.09% (w/v) glucose, and a buffer. In some embodiments, the formulation comprises about 5% (v/v) DMSO, about 2.5% (w/v) albumin, about 0.6% (w/v) glucose, and a buffer. In some embodiments, the formulation comprises about 4-10% (v/v) DMSO, about 2-8% (w/v) albumin, about 0-1.5% (w/v) glucose, and a buffer. In some embodiments, the formulation comprises about 4-6% (v/v) DMSO, about 2-8% (w/v) albumin, about 0-1.5% (w/v) glucose, and a buffer. In some embodiments, the formulation comprises about 5% (v/v) DMSO, about 2.5% (w/v) albumin, about 0.6% (w/v) glucose, and a buffer. In some embodiments, the formulation comprises about 5% (v/v) DMSO, about 7.5% (w/v) albumin, about 0.6% (w/v) glucose, and a buffer.

This disclosure further contemplates any of the foregoing formulations comprising about 0%-5% (w/v) glucose or comprising any one of the ranges specified herein, such as in the preceding paragraphs.

In some embodiments, the formulations provided herein further comprise at least one further excipient. In some embodiments, the formulation comprises about 1 mM-500 mM. about 5 mM-400 mM, about 10 mM-300 mM, about 25mN-250 mM, about 50 mM-200 mM of the at least one further excipient. In some embodiments, the formulation comprises about 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, 100 mM, 101 mM, 102 mM, 103 mM, 104 mM, 105 mM, 106 mM, 107 mM, 108 mM, 109 mM, 110 mM, 111 mM, 112 mM, 113 mM, 114 mM, 115 mM, 116 mM, 117 mM, 118 mM, 119 mM, 120 mM, 121 mM, 122 mM, 123 mM, 124 mM, 125 mM, 126 mM, 127 mM, 128 mM, 129 mM, 130 mM, 131 mM, 132 mM, 133 mM, 134 mM, 135 mM, 136 mM, 137 mM, 138 mM, 139 mM, 140 mM, 141 mM, 142 mM, 143 mM, 144 mM, 145 mM, 146 mM, 147 mM, 148 mM, 149 mM, 150 mM, 151 mM, 152 mM, 153 mM, 154 mM, 155 mM, 156 mM, 157 mM, 158 mM, 159 mM, 160 mM, 161 mM, 162 mM, 163 mM, 164 mM, 165 mM, 166 mM, 167 mM, 168 mM, 169 mM, 170 mM, 171 mM, 172 mM, 173 mM, 174 mM, 175 mM, 176 mM, 177 mM, 178 mM, 179 mM, 180 mM, 181 mM, 182 mM, 183 mM, 184 mM, 185 mM, 186 mM, 187 mM, 188 mM, 189 mM, 190 mM, 191 mM, 192 mM, 193 mM, 194 mM, 195 mM, 196 mM, 197 mM, 198 mM, 199 mM, 200 mM, 201 mM, 202 mM, 203 mM, 204 mM, 205 mM, 206 mM, 207 mM, 208 mM, 209 mM, 210 mM, 211 mM, 212 mM, 213 mM, 214 mM, 215 mM, 216 mM, 217 mM, 218 mM, 219 mM, 220 mM, 221 mM, 222 mM, 223 mM, 224 mM, 225 mM, 226 mM, 227 mM, 228 mM, 229 mM, 230 mM, 231 mM, 232 mM, 233 mM, 234 mM, 235 mM, 236 mM, 237 mM, 238 mM, 239 mM, 240 mM, 241 mM, 242 mM, 243 mM, 244 mM, 245 mM, 246 mM, 247 mM, 248 mM, 249 mM, or 250 mM of the at least one further excipient. In some embodiments, the at least one further excipient is selected from the group consisting of taurine, trehalose, sucrose, lactose, propanediol, mannitol, sorbitol, proline, glutathione, glycine, alanine, or lysine. In some embodiments, the at least one further excipient is taurine. In some embodiments, the at least one further excipient is trehalose. In some embodiments, the at least one further excipient is 1,2 propanediol or 1,3 propanediol. In some embodiments, the at least one further excipient is propylene glycol.

In some embodiments, the formulations comprise a polymeric excipient. In some embodiments, the formulations comprise about 1%-20% of the polymeric excipient. In some embodiments, the formulations comprise about 3%-18%, about 5%-15%, or about 7.5%-12.5%, of a polymeric excipient. In some embodiments, the formulations comprise about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, or about 20% of a polymeric excipient. In some embodiments, the polymeric excipient is selected from the group consisting of poly(ethylene glycol) (PEG), dextran, and poly(vinyl pyrrolidone) (PVP). In some embodiments, the polymeric excipient is dextran. In some embodiments, the formulations do not comprise a polymeric excipient. In some embodiments, the formulations do not comprise dextran.

### Cell Preparations

Some aspects of this disclosure provide cell preparations comprising a population of cells or a tissue in a cryopreservative formulation as provided herein. In some embodiments, the cell preparation is cryopreserved. The cell preparation provided herein may be cryopreserved by being cryogenically frozen at any appropriate temperature known in the art. For example, in some embodiments, the cell preparations provided herein are cryopreserved at temperatures between −100° C. and −200° C. In some embodiments the cell preparations provided herein are cryopreserved at temperatures below −125° C. In some embodiments the cell preparations provided herein are cryopreserved at temperatures at or below −135° C. In some embodiments the cell preparations provided herein are cryopreserved at temperatures below −150° C.

The cryopreservative formulations and cell preparations provided herein can be used in connection with different cell types and with different administration sites. While ophthalmologic applications are preferred, other applications are also contemplated and embraced by the present disclosure. In some embodiments, the population of cells is selected from retinal pigment epithelium (RPE) cells, photoreceptor cells and/or progenitor cells thereof, photoreceptor rescue cells and corneal endothelial cells and/or progenitor cells thereof.

In some embodiments, the cell preparation is suitable for transplantation into a subject once thawed, optionally without being diluted. In some embodiments, the cell preparation is suitable for transplantation into a subject once thawed and diluted with a diluent. In some embodiments, the diluent is GS2, GS2 Plus, BSS, BSS Plus®, a dextran and HSA solution, PBS, DMEM, MEM, albumin, or any other appropriate diluent. In some embodiments, the diluent is GS2 or GS2 Plus. In some embodiments, the diluent is a solution comprising (a) a buffer that maintains the solution at a physiological pH; (b) at least 2 mM glucose; and (c) an osmotically active agent maintaining the solution at a physiological osmolarity. In some embodiments, the diluent is GS2 or GS2 Plus. In some embodiments, the diluent is a solution comprising (a) a buffer that maintains the solution at a physiological pH; (b) at least 2 mM glucose; and (c) an osmotically active agent maintaining the solution at an osmolarity that is higher than physiological osmolarity, for example in the range of 350-450 mOsm/kg or an osmolality in the range of 300-1250 mOsm/kg. In some embodiments, the diluent further comprises divalent cations. In some embodiments, divalent cations comprise calcium and/or magnesium. In some embodiments, the diluent comprises calcium. In some embodiments, the diluent further comprises magnesium. In some embodiments, the diluent comprises an acetate buffer and/or a citrate buffer. In some embodiments, the diluent is a solution which combines two or more or any number of criteria (e.g., pH, osmolarity, solutes (buffer, glucose, osmotically active agent, magnesium, calcium, potassium, polymer), concentrations, etc.). In some embodiments, the diluent is a solution comprising a buffering agent, glucose, and an osmotically active agent with or without added polymer, a solution comprising potassium and a solution not comprising potassium, as well as a solution comprising any combination of solutes at any concentration provided for the respective solute. It will also be understood that the disclosure embraces a solution comprising the listed solutes as well as a solution essentially consisting of or consisting of the listed solutes and a solvent, e.g., water. These alternatives are not spelled out here for purposes of brevity.

For example, in some embodiments, the diluent is a solution comprising or consisting essentially of calcium chloride, magnesium chloride, sodium citrate, sodium chloride, and glucose, e.g., D-glucose, in water. In some embodiments, the diluent is a solution comprising or consisting essentially of calcium chloride, magnesium chloride, sodium citrate, sodium chloride, glucose, e.g., D-glucose, and potassium chloride, in water. In some embodiments, the diluent is GS2 or GS2 Plus, as described in WO2017/031312A1, the contents of which are incorporated by reference herein.

In some embodiments, the diluent is a solution comprising about 0.1 to about 1.2 mM CaCl₂, about 0.05 to about 5 mM MgCl₂, about 1 to about 2.5 mM KCl, about 0.5 to about 2 mM sodium citrate, about 14 to about 17 mM dextrose, and about 125 to about 175 mM NaCl. In some embodiments, the diluent is a solution comprising about 0.9 mM CaCl₂, about 0.3 mM MgCl₂, about 2 mM KCl, about 1.2 mM sodium citrate, about 15 mM dextrose, and about 145 mM NaCl. The diluent may further comprise sodium acetate. The diluent may further comprise a polymer such as hyaluronic acid or a solvate thereof such as sodium hyaluronate, optionally at a concentration of about 0.01 to about 0.05% (w/v), including about 0.05% (w/v). It may further comprise buffering components such as sodium phosphate dibasic heptahydrate and/or sodium phosphate monobasic monohydrate and/or sodium phosphate monobasic dihydrate.

In some embodiments, the diluent is a solution comprising about 0.008% to about 0.012% CaCl₂ dihydrate, about 0.0048% to about 0.0072% MgCl₂ hexahydrate, about 0.012% to about 0.018% KCl, about 0.028% to about 0.042% sodium citrate dihydrate, about 0.23% to about 0.35% dextrose, and about 0.68% to about 1.02% NaCl. In some embodiments, the diluent is a solution that comprises about 0.01% CaCl₂ dihydrate, about 0.006% MgCl₂ hexahydrate, about 0.015% KCl, about 0.035% sodium citrate dihydrate, at least 0.25% dextrose, and about 0.85% NaCl. The diluent may further comprise sodium acetate. The diluent may further comprise a polymer such as hyaluronic acid or a solvate thereof such as sodium hyaluronate, optionally at a concentration of about 0.01 to about 0.05% (w/v), including about 0.05% (w/v). It may further comprise buffering components such as sodium phosphate dibasic heptahydrate and/or sodium phosphate monobasic monohydrate and/or sodium phosphate monobasic dihydrate.

In some embodiments, the diluent comprises 5% dextrose, 0.9% NaCl, BSS® sterile irrigating solution, 0.1 N NaOH, and 0.9% NaCl irrigation. In some embodiments, the diluent comprises 3% sodium hyaluronate, 5% dextrose anhydrous, sodium phosphate dibasic dodecahydrate, BSS® sterile irrigating solution (e.g., 0.64% NaCl, 0.075% KCl, 0.048% CaCl₂ dihydrate, 0.03% MgCl₂ hexahydrate, 0.39% sodium acetate trihydrate, and 0.17% sodium citrate dihydrate).

In some embodiments, the diluent comprises about 0.27% glucose, about 0.84% sodium chloride, about 0.016% potassium chloride, about 0.01% calcium chloride, about 0.006% magnesium chloride, about 0.036% sodium citrate, and optionally sodium acetate, (e.g., at about 0.08%), optionally sodium hyaluronate (e.g., at about 0.049%), and optionally sodium phosphate dibasic heptahydrate (e.g., at about 0.0007%) and sodium phosphate monobasic monohydrate (e.g., at about 0.0001%). In some embodiments, the diluent comprises about 15 mM glucose, about 144 mM sodium chloride, about 2.1 mM potassium chloride, about 0.9 mM calcium chloride, about 0.3 mM magnesium chloride, about 1.2 mM sodium citrate, optionally sodium acetate (e.g., at about 6 mM), optionally sodium hyaluronate, and optionally sodium phosphate dibasic heptahydrate (e.g., at about 0.027 mM) and sodium phosphate monobasic monohydrate (e.g., at about 0.007 mM). Such diluents include GS2.

In some embodiments, the diluent comprises about 0.27% glucose, about 0.84% sodium chloride, about 0.016% potassium chloride, about 0.01% calcium chloride, about 0.006% magnesium chloride, about 0.036% sodium citrate, and optionally sodium acetate (e.g., at about 0.08%), optionally sodium hyaluronate (e.g., at about 0.049%), and optionally sodium phosphate monobasic dihydrate (e.g., at about 0.047%). In some embodiments, the diluent comprises about 15 mM glucose, about 144 mM sodium chloride, about 2.1 mM potassium chloride, about 0.9 mM calcium chloride, about 0.3 mM magnesium chloride, about 1.2 mM sodium citrate, optionally sodium acetate (e.g., at about 6 mM), optionally sodium hyaluronate, and optionally sodium phosphate monobasic dihydrate (e.g., at about 3 mM). Such diluents include GS2 Plus.

In some embodiments, the cell preparations provided herein are formulated for administration to a subject, for example, for administration via injection, once thawed and diluted. The cell preparations provided herein may comprise a population of cells such as RPE cells and/or progenitors thereof, photoreceptor cells and/or progenitors thereof, and corneal endothelial cells and/or progenitor cells thereof and photoreceptor rescue cells.

Exemplary cell or tissue preparations, post-thaw may be formulated to be suitable for use in treating a human patient, e.g., pyrogen-free or essentially pyrogen-free, pathogen-free, sterile, and at physiological pH and osmolarity. In some embodiments, the preparations provided herein are formulated for injection into a specific site, e.g., in the case of ophthalmologic preparations for treating retinal diseases or disorders, into the vitreous humor for delivery to the site of retinal or choroidal damage.

Cell preparations provided by the present disclosure may include additionally therapeutic agents, for example, an immunosuppressant, a pro-angiogenic agent, or nutrients or growth factors supporting survival and/or implantation of the cells in the preparation.

The volume and the number of cells in the cell preparations to be administered will depend on the specific application. Typically, for cell transplantation applications, it is desirable to reduce the volume administered as much as possible. Accordingly, the cell preparations may be formulated so that minimized volumes may be delivered. Cell concentrations for injection may be at any concentration that is effective and non-toxic. In some embodiments, the volume of the cell preparation to be administered is between 1 to 1000 μL. In some embodiments, the volume of the cell preparation to be administered is between 1 to 50 μl, or between 10 to 50 μl or between 25 to 50 μl or between 50 to 100 μl or between 50 to 200 μl or between 50 to 300 μl or between 50 to 400 μl or between 50 to 500μl, or between 100 to 500 μl, or between 100 to 400 μl or between 100 to 300 μl, or between 100 to 300 μl or about 200 μl or about 150 μl. In some embodiments the volume of the cell preparation to be administered is about 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 900, 950 or 1000 μl.

In some embodiments, the number of cells and/or the concentration of cells in a cell preparation provided herein may be determined by counting viable cells and excluding non-viable cells. For example, non-viable cells may be detected by failure to exclude a vital dye (such as Trypan Blue), or using a functional assay (such as the ability to adhere to a culture substrate, phagocytosis, etc.). Additionally, the number of cells or the concentration of cells of a desired cell type may be determined by counting cells that express one or more cell markers characteristic of that cell type and/or excluding cells that express one or more markers indicative of a cell type other than the desired cell type.

In some embodiments, a cell preparation to be administered comprises about or at least 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ cells, or more cells.

In some embodiments, the number of cells per vial is about 10⁵ to 10⁷ cells per vial prior to cryopreservation. In some embodiments, the number of cells per vial is about 450,000 to 1,500,000 cells per vial prior to cryopreservation. In some embodiments, the number of cells per vial is about 1,750,000 to about 3,500,000 cells per vial prior to cryopreservation. In some embodiments, the number of cells per vial is about 10⁵ to 10⁷ cells per vial following cryopreservation. In some embodiments, the number of cells per vial is about 450,000 to 1,500,000 cells per vial following cryopreservation, thawing and dilution. In some embodiments, the number of cells per vial is about 1,750,000 to about 3,500,000 cells per vial prior following cryopreservation, thawing and diluting.

The afore-mentioned numbers of cells may be present in the cryopreserved cell preparation in a single cryovial. The cells may be present in about 10 to about 500 μL of cryoprotective formulation, including in about 10 to about 200 μL of cryoprotective formulation, or in about 10 to about 100 μL of cryoprotective formulation, or about 10 to about 50 μL of cryoprotective formulation, or about 20 to about 50 μL of cryoprotective formulation.

In some embodiments, the population of cells is suitable for transplantation into the eye of a subject. In some embodiments, the population of cells suitable for transplantation to the eye of a subject comprises RPE cells, RPE progenitor cells, iris pigmented epithelial (IPE) cells, and other vision associated neural cells, such as internuncial neurons (e.g., “relay” neurons of the inner nuclear layer (INL)) and amacrine cells, retinal cells, rods, cones, and corneal cells (e.g. corneal endothelial cells), neural cells, neural progenitor cells, neural stem cells, photoreceptor cells (e.g. photoreceptor progenitor cells), photoreceptor rescue cells and mesenchymal cells, such as, e.g., mesenchymal stem cells (MSCs). The cells so formulated may be generated by directed differentiation of pluripotent or multipotent stem cells, including induced human pluripotent stem cells (hiPSC), human embryonic stem cells (hESC) and somatic cells (including transdifferentiated cells and stem cells).

In some embodiments, the cell preparations comprise a population of RPE cells in a cryopreservative formulation provided herein. Suitable RPE cells may be differentiated from pluripotent stem cells, such as human embryonic stem(ES) cells or induced pluripotent stem cells (iPS cells), and be molecularly distinct from embryonic stem cells, iPS cells, adult-derived RPE cells, and fetal-derived RPE cells. In some embodiments, adult-derived RPE cells, and fetal-derived RPE cells are used.

Where ES cell derived RPE cells are used, the cell preparation, in some embodiments, does not comprise a detectable amount of residual ES cells, such that the cell preparations provided herein do not pose an unacceptable risk to a recipient of such preparation. Where iPS cell derived RPE cells are used, the cell preparation, in some embodiments, does not comprise a detectable amount of residual iPS cells, such that the cell preparations provided herein do not pose an unacceptable risk to a recipient of such preparation.

In some embodiments, the cell preparations comprise a population of photoreceptor rescue cells in a cryopreservative formulation provided herein. Suitable photoreceptor rescue cells may be differentiated from pluripotent stem cells, such as human embryonic stem cells or iPS cells, and be molecularly distinct from embryonic stem cells, iPS cells, adult-derived photoreceptor rescue cells, and fetal-derived photoreceptor rescue cells. In some embodiments, adult-derived photoreceptor rescue cells, and fetal-derived photoreceptor rescue cells are used.

Where ES cell derived photoreceptor rescue cells are used, the cell preparation, in some embodiments, does not comprise a detectable amount of residual ES cells, such that the cell preparations provided herein do not pose an unacceptable risk to a recipient of such preparation. Where iPS cell derived photoreceptor rescue cells are used, the cell preparation, in some embodiments, does not comprise a detectable amount of residual iPS cells, such that the cell preparations provided herein do not pose an unacceptable risk to a recipient of such preparation.

In some embodiments, the cell preparations comprise a population of corneal endothelial cells in a cryopreservative formulation provided herein. Suitable corneal endothelial cells may be differentiated from pluripotent stem cells, such as human embryonic stem cells or iPS cells, and be molecularly distinct from embryonic stem cells, iPS cells, adult-derived corneal endothelial cells, and fetal-derived corneal endothelial cells. In some embodiments, adult-derived corneal endothelial cells, and fetal-derived corneal endothelial cells are used.

Where ES cell derived corneal endothelial cells are used, the cell preparation, in some embodiments, does not comprise a detectable amount of residual ES cells, such that the cell preparations provided herein do not pose an unacceptable risk to a recipient of such preparation. Where iPS cell derived corneal endothelial cells are used, the cell preparation, in some embodiments, does not comprise a detectable amount of residual iPS cells, such that the cell preparations provided herein do not pose an unacceptable risk to a recipient of such preparation.

In some embodiments, the cell preparation comprising a population of cells suitable for transplantation into the eye of a subject is suitable for injection into the eye of the subject. In some embodiments, such a cell preparation may be used for treating retinal degeneration diseases or disorders, including, but not limited to, retinal detachment, retinal dysplasia, Angioid streaks, Myopic Macular Degeneration, or retinal atrophy or associated with a number of vision-altering ailments that result in photoreceptor damage and blindness, such as, for example, choroideremia, diabetic retinopathy, macular degeneration (e.g., age-related macular degeneration), retinitis pigmentosa, and Stargardt's Disease (fundus flavimaculatus).

The RPE cells may be stable, terminally differentiated RPE cells that do not de-differentiate to a non-RPE cell type. The RPE cells described herein may be functional RPE cells, characterized by the ability to integrate into the retina upon corneal, sub-retinal, or other administration into a human or a non-human animal.

The RPE cells may express RPE cell markers. For example, the level of expression of markers such as RPE65, PAX2, PAX6, tyrosinase, bestrophin, PEDF, CRALBP, Otx2, and/or MITF may be equivalent to that in naturally occurring RPE cells. The level of maturity of the RPE cells may be assessed by measuring expression of at least one of PAX2, PAX6, and tyrosinase, or their respective expression levels.

In some embodiments, the RPE cells comprised in a pharmaceutical composition provided herein may be identified and characterized based on the degree of pigmentation of the cell. Changes in pigment can be controlled by the density at which the RPE cells are cultured and maintained and the duration of time for which RPE are maintained in culture. Differentiated RPE cells that are rapidly dividing are more lightly pigmented. In contrast, more slowly dividing or non-dividing RPE adopt their characteristic polygonal or hexagonal shape and increase pigmentation level by accumulating melanin and lipofuscin. For example, quiescent RPE cultures (e.g., due to confluence) typically increase their level of pigmentation over time. As such, accumulation of pigmentation serves as an indicator of RPE differentiation and increased pigmentation associated with cell density serves as an indicator of RPE maturity. For example, mature RPE cells may be subcultured at a lower density, such that the pigmentation decreases. In this context, mature RPE cells may be cultured to produce less mature RPE cells. Such RPE cells are still differentiated RPE cells that express markers of RPE differentiation.

For example, in some embodiments, a preparation is provided that comprises RPE cells the average melanin content of which is less than 8 pg/cell, less than 7 pg/cell, less than 6 pg/cell, or less than 5 pg/cell, e.g., between 0.1-8 pg/cell, between 0.1-7 pg/cell, between 0.1-6 pg/cell, between 0.1-5 pg/cell, between 0.1-4 pg/cell, between 0.1-3 pg/cell, between 0.1-2 pg/cell, between 0.1-1 pg/cell, between 1-8 pg/cell, between 1-7 pg/cell, between 1-6 pg/cell, between 1-5 pg/cell, between 1-4 pg/cell, between 1-3 pg/cell, between 1-2 pg/cell, between 2-6 pg/cell, between 3-5 pg/cell, or between 4-5 pg/cell, such as, for example, 4.2-4.8 pg/cell, or between 0.1-5 pg/cell. In a further example, the average melanin content may be less than 5 pg/cell, e.g., between 0.1-5 pg/cell, between 0.2-5 pg/cell, 0.5-5 pg/cell, 1-5 pg/cell, 2-5 pg/cell, 3-5 pg/cell, 4-5 pg/cell, or 4.5-5 pg/cell.

In some embodiments, a preparation comprising RPE cells in a cell cryopreservative formulation described herein, is provided, wherein the RPE cells maintain their phenotype following transplantation of the RPE cells to a subject, e.g., following injection of the preparation into the eye of the subject. The RPE cells may maintain their phenotype for the lifespan of the recipient after transplantation. For example, the RPE cells may maintain their phenotype following transplantation for at least about 1, 2, 3, 4, 5, 6, 7, 8, 9, 10, 11, 12, 13, 14, 15, 16, 17, 18, 19, or 20 days. Further, the RPE cells may maintain their phenotype following transplantation for at least about 1, 2, 3, 4, 5, 6, 7, 8, 9, or 10 weeks. The RPE cells may maintain their phenotype following transplantation for at least about 1, 2, 3, 4, 5, 6, 7, 8, 9, 10, 11, or 12 months. The RPE cells may maintain their phenotype following transplantation for at least about 1, 2, 3, 4, 5, 6, 7, 8, 9, 10, 11, 12, 13, 14, 15, 16, 17, 18, 19, 20, or more years.

Cells may be formulated in a preparation as provided herein for delivery in a pharmaceutically acceptable ophthalmic vehicle, such that the preparation is maintained in contact with the ocular surface for a sufficient time period to allow the cells to penetrate the affected regions of the eye, as for example, the anterior chamber, posterior chamber, vitreous body, aqueous humor, vitreous humor, cornea, iris/ciliary, lens, choroid, retina, sclera, suprachoridal space, conjunctiva, subconjunctival space, episcleral space, intracorneal space, epicorneal space, pars plana, surgically-induced avascular regions, or the macula. Cells may have several possible arrangements such as individual cells, clusters, spheres, aggregates, sheets, or any combination thereof, which may be contained in an aqueous vehicle, gel, matrix, polymer or the like.

In some embodiments, a preparation is provided herein, in which cells are contained in a sheet of cells. For example, RPE cells or corneal endothelial cells or photoreceptor cells or photoreceptor rescue cells may be contained in a sheet. For example, a sheet of cells comprising RPE cells may be prepared by culturing RPE cells on a substrate from which an intact sheet of cells can be released, e.g., a thermoresponsive polymer such as a thermoresponsive poly(N-isopropylacrylamide) (PNIPAAm)-grafted surface, upon which cells adhere and proliferate at the culture temperature, and then upon a temperature shift, the surface characteristics are altered causing release of the cultured sheet of cells (e.g., by cooling to below the lower critical solution temperature (LCST) (see da Silva et al., Trends Biotechnol. 2007 December;25(12):577-83; Hsiue et al., Transplantation. 2006 February 15;81(3):473-6; Ide, T. et al. (2006); Biomaterials 27, 607-614, Sumide, T. et al. (2005), FASEB J. 20, 392-394; Nishida, K. et al. (2004), Transplantation 77, 379-385; and Nishida, K. et al. (2004), N. Engl. J. Med. 351, 1187-1196 each of which is incorporated by reference herein in its entirety). The sheet of cells may be adherent to a substrate suitable for transplantation, such as a substrate that may dissolve in vivo when the sheet is transplanted into a host organism, e.g., prepared by culturing the cells on a substrate suitable for transplantation, or releasing the cells from another substrate (such as a thermoresponsive polymer) onto a substrate suitable for transplantation. An exemplary substrate potentially suitable for transplantation may comprise gelatin (see Hsiue et al., supra). Alternative substrates that may be suitable for transplantation include fibrin-based matrixes and others. The sheet of cells may be used in the manufacture of a medicament for the prevention or treatment of a disease of retinal degeneration.

In some embodiments, a preparation is provided herein, in which cells are in clusters, spheres, or aggregates. In some embodiments, cells provided herein are administered in combination with a biomaterial, for example a biomaterial that can be polymerized.

In some embodiments, cells provided herein (e.g., RPE cells) are administered as an implant or device. In certain embodiments, the device is a bioerodible implant for treating a medical condition of the eye comprising an active agent dispersed within a biodegradable polymer matrix, wherein at least about 75% of the particles of the active agent have a diameter of less than about 10 pm. The bioerodible implant is sized for implantation in an ocular region. The ocular region can be any one or more of the anterior chamber, the posterior chamber, the vitreous cavity, the choroid, the suprachoroidal space, the conjunctiva, the subconjunctival space, the episcleral space, the intracorneal space, the epicorneal space, the sclera, the pars plana, surgically-induced avascular regions, the macula, and the retina. The biodegradable polymer can be, for example, a poly(lactic-co-glycolic)acid (PLGA) copolymer. In certain embodiments, the ratio of lactic to glycolic acid monomers in the polymer is about 25/75, 40/60, 50/50, 60/40, 75/25 weight percentage, more preferably about 50/50. Additionally, the PLGA copolymer can be about 20, 30, 40, 50, 60, 70, 80 to about 90 percent by weight of the bioerodible implant. In certain preferred embodiments, the PLGA copolymer can be from about 30 to about 50 percent by weight, preferably about 40 percent by weight of the bioerodible implant. Cells provided herein (e.g., RPE cells, photoreceptor cells, photoreceptor rescue cells and corneal endothelial cells) can be transplanted in conjunction with a biocompatible polymer such as polylactic acid, poly(lactic-glycolic acid), PDLGA 50:50, Pd Lg A 85:15, and biodegradable membrane INION GTR® (mixture of biocompatible polymers). See U.S. Pat. Nos. 6,331,313, 7,462,471 and 7,625,582. See also Hutala, et al. (2007) “In vitro biocompatibility of degradable biopolymers in cell line cultures from various ocular tissues: Direct contact studies”. Journal of Biomedical Materials Research 83A (2): 407-413; Lu et al. (1998) J Biomater Sci Polym Ed 9: 1187 205; and Tomita et al. (2005) Stem Cells 23: 1579-88. In some instances the biodegradable material can be polymerized.

The volume of a pharmaceutical composition provided by some embodiments of this disclosure depends on factors such as the mode of administration, number of cells to be delivered, age and weight of the patient, and type and severity of the disease being treated. For example, if administered by injection, the volume of a pharmaceutical composition of cells of the disclosure may be about 1-1000 μL. In some embodiments, the volume may be about 1-200 μL. For example, the volume of a composition of the disclosure may be about 10-50, 20-50, 25-50, or 1-200 μL. The volume of a composition of the disclosure may be about 10, 20, 30, 40, 50, 100, 110, 120, 130, 140, 150, 160, 170, 180, 190, 200, 250, 300,350, 400, or 450 μL, or higher.

In some embodiments, the concentration of cells in the cryopreservative formulation (or the cryopreserved cell preparation) is about 300, 500, 1×10³, 1.5×10³, 2×10³, 2.5×10³, 3×10³, 3.333×10³, 3.5×10³, 4×10³, 4.4 ×10³, 5×10³, 5.5×10³, 7.5×10³, 1×10⁴, 1.5×10⁴, 2×10⁴, 2.5×10⁴, 3×10⁴, 3.5×10⁴, 4×10⁴, 4.5×10⁴, 5×10⁴, 5.5×10⁴, 6×10 ⁴, 6.5×10⁴, 7×10⁴, 7.5×10⁴, 8×10⁴, 8.5×10⁴, 9×10⁴, 9.5×10⁴, 1×10⁵, 1.05×10⁵, 1.1×10⁵, 1.15×10⁵, 1.2×10⁵, 1.25×10⁵, 1.3×10⁵, 1.35×10⁵, 1.4×10⁵, 1.45×10⁵, or 1.5 ×10⁵ cells/μL. In some embodiments, the concentration of cells in the cryopreservative formulation (or the cryopreserved cell preparation) is about 2,000-80,000 cells/μL, 2,000-70,000 cells/μL, 3,000-70,000 cells/μL, 3,000-60,000 cells/μL, 3,000-50,000 cells/μL, 3,000-10,000 cells/μL, 3,000-5,000 cells/μL, 3,000-4,000 cells/μL, 3,500 cells/μL, 10,000-100,000 cells/μL, 10,000-90,000 cells/μL, 10,000-80,000 cells/μL, 10,000-70,000 cells/μL, 10,000-60,000 cells/μL, 10,000-50,000 cells/μL, 10,000-40,000 cells/μL, 10,000-30,000 cells/μL, 10,000-20,000 cells/μL, 20,000-100,000 cells/μL, 20,000-90,000 cells/μL, 20,000-80,000 cells/μL, 20,000-70,000 cells/μL, 20,000-60,000 cells/μL, 20,000-50,000 cells/μL, 20,000-40,000 cells/μL, 20,000-30,000 cells/μL, 30,000-100,000 cells/μL, 30,000-90,000 cells/uL, 30,000-80,000 cells/μL, 30,000-70,000 cells/μL, 30,000-60,000 cells/μL, 30,000-50,000cells/μL, 30,000-40,000 cells/μL, 40,000-100,000 cells/μL, 40,000-90,000 cells/μL, 40,000-80,000 cells/μL, 40,000-70,000 cells/μL, 40,000-60,000 cells/μL, 40,000-50,000 cells/μL, 50,000-100,000 cells/μL, 50,000-90,000 cells/μL, 50,000-80,000 cells/μL, 50,000-70,000cells/μL, 50,000-60,000 cells/μL, 60,000-100,000 cells/μL, 60,000-90,000 cells/μL, 60,000-80,000 cells/μL, 60,000-70,000 cells/μL, 70,000-100,000 cells/μL, 70,000-90,000 cells/μL, 70,000-80,000 cells/μL, 80,000-100,000 cells/μL, 80,000-90,000 cells/μL, 90,000-100,000 cells/μL, 10,000-150,000 cells/μL, 20,000-150,000 cells/μL, 30,000-150,000 cells/μL, 40,000-150,000 cells/μL, 50,000-150,000 cells/μL, 60,000-150,000 cells/μL, 70,000-150,000 cells/μL, 80,000-150,000 cells/μL, 90,000-140,000 cells/μL, 100,000-150,000 cells/μL, 100,000-130,000 cells/μL, 120,000-150,000 cells/μL, or 120,000-125,000 cells/μL.

In some embodiments, the preparation supports survival of the cells in the population of cells during storage of the preparation. In some embodiments, the preparation supports survival of the cells during storage for at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, at least 1 year, at least 2 years, at least 3 years, at least 4 years at least 5 years or more. In some embodiments, the preparation supports survival of the cells during storage for about 1 week, about 2 weeks, about 3 weeks, about 4 weeks, about 5 weeks, about 6 weeks, about 7 weeks, about 8 weeks, about 1 week to about 8 weeks, about 1 week to about 4 weeks, about 1 week to about 2 weeks. In some embodiments, the preparation supports survival of the cells during storage for about 1 day, about 2 days, about 3 days, about 4 days, about 5 days, about 6 days, or about 7 days, or about 1 day to about 7 days, about 1 day to about 5 days, about 1 day to about 3 days or about 1 day to about 2 days. The preparation may be stored at temperatures ranging from about −100 to about −200° C., or about −135° C. to about −200° C., without limitation.

In some embodiments, at least 30% of the cells in the cell population are viable after about 1-10 years, about 1-9 years, about 1-8 years, about 1-7 years about 1-6 years, about 1-5 years, about 1-4 years, about 1-3 years or about 1-2 years or less of storage of the preparation at −100 to −200° C., preferably at or less than −135° C. In some embodiments, at least 40% of the cells in the cell population are viable after about 1-10 years, about 1-9 years, about 1-8 years, about 1-7 years about 1-6 years, about 1-5 years, about 1-4 years, about 1-3 years or about 1-2 years or less of storage of the preparation at −100 to −200° C., preferably at or less than −135° C. In some embodiments, at least 50% of the cells in the cell population are viable after about 1-10 years, about 1-9 years, about 1-8 years, about 1-7 years about 1-6 years, about 1-5 years, about 1-4 years, about 1-3 years or about 1-2 years or less of storage of the preparation at −100 to −200° C., preferably at or less than −135° C. In some embodiments, at least 55% of the cells in the cell population are viable after about 1-10 years, about 1-9 years, about 1-8 years, about 1-7 years about 1-6 years, about 1-5 years, about 1-4 years, about 1-3 years or about 1-2 years or less of storage of the preparation at −100 to −200° C., preferably at or less than −135° C. In some embodiments, at least 60% of the cells in the cell population are viable after about 1-10 years, about 1-9 years, about 1-8 years, about 1-7 years about 1-6 years, about 1-5 years, about 1-4 years, about 1-3 years or about 1-2 years or less of storage of the preparation at −100 to −200° C., preferably at or less than −135° C. In some embodiments, at least 65% of the cells in the cell population are viable after about 1-10 years, about 1-9 years, about 1-8 years, about 1-7 years about 1-6 years, about 1-5 years, about 1-4 years, about 1-3 years or about 1-2 years or less of storage of the preparation at −100 to −200° C., preferably at or less than −135° C. In some embodiments, at least 70% of the cells in the cell population are viable after about 1-10 years, about 1-9 years, about 1-8 years, about 1-7 years about 1-6 years, about 1-5 years, about 1-4 years, about 1-3 years or about 1-2 years or less of storage of the preparation at −100 to −200° C., preferably at or less than −135° C. In some embodiments, at least 80% of the cells in the cell population are viable after about 1-10 years, about 1-9 years, about 1-8 years, about 1-7 years about 1-6 years, about 1-5 years, about 1-4 years, about 1-3 years or about 1-2 years or less of storage of the preparation at −100 to −200° C., preferably at or less than −135° C. In some embodiments, at least 90% of the cells in the cell population are viable after about 1-10 years, about 1-9 years, about 1-8 years, about 1-7 years about 1-6 years, about 1-5 years, about 1-4 years, about 1-3 years or about 1-2 years or less of storage of the preparation at −100 to −200° C., preferably at or less than −135° C. In some embodiments, the preparation supports maintenance of the plating efficiency of the population of cells during storage of the preparation. In some embodiments, after about 1-10 years, about 1-9 years, about 1-8 years, about 1-7 years about 1-6 years, about 1-5 years, about 1-4 years, about 1-3 years or about 1-2 years or less of storage of the preparation at −100 to −200° C., preferably at or less than −135° C., the population of cells exhibits at least 50%, at least 55%, at least 60%, at least 65%, at least 70%, at least 75%, at least 80%, at least 85%, at least 90%, at least 95%, at least 97%, at least 98%, or at least 99% of its original plating efficiency, wherein the original plating efficiency refers to the plating efficiency of the population of cells at the beginning of the storage period. In some embodiments, the preparation is within a storage container. In some embodiments, the storage container is a vial, ampule, bottle, syringe, a cooler package, or other suitable storage container. In some embodiments, the preparation is within a vial. In some embodiments, the preparation is within a syringe.

The cell preparations provided herein, post-thaw, are suitable for administration to a subject following minimal processing (e.g. dilution). In some embodiments, the preparation consists essentially of cells, a cell population, or a tissue and a cryopreservative formulation as provided herein. In some embodiments, the preparation comprises one or more pharmaceutically active ingredients, for example, a preservative, an antioxidant, a radical scavenger, an immunosuppressant, a pro-angiogenic factor, an anti-angiogenic factor, a hormone (e.g., a growth hormone), or a cell nutrient or substrate supporting cell growth, survival, and implantation.

Preservatives may include but are not limited to benzalkonium chloride (BAK), benzethonium chloride, chlorobutanol, phenylmercuric acetate or nitrate, thimerosal, polyquaternium-1 (POLYQUAD®), stabilized oxychloro complex (PURITE®) or methyl or propylparabens. Antioxidants and/or radical scavengers may include but are not limited to vitamin C, vitamin E, β-carotene, conenzyme Q-10, selenium, thioredoxin reductase, glutathione, α-tocopherol, L-cysteine and N-acetylcysteine (NAC), Ascorbic acid-2-phosphate (AAP), zinc (e.g., zinc oxide), copper (e.g., copper oxide), dimethyl sulfoxide (DMSO), glycerol, mannitol, and polyphenolic compounds. Immunosuppressants may include but are not limited to anti-lymphocyte globulin (ALG) polyclonal antibody, anti-thymocyte globulin (ATG) polyclonal antibody, azathioprine, BASILIXIMAB® (anti-IL-2Rα receptor antibody), cyclosporin (cyclosporin A), DACLIZUMAB® (anti-IL-2Rα receptor antibody), everolimus, mycophenolic acid, RITUXIMAB® (anti-CD20 antibody), sirolimus, and tacrolimus, mesenchymal stem cells, and mycophenolate mofetil. Pro-angiogenic factors may include but are not limited to VEGF, HGF, KGF, bFGF, TIMP1, ANG2, PDGF-bb, TPO, HB-EGF, TGF-β1, MMP-9, and MMP-2. Anti-angiogenic factors may include but are not limited to somatostatin, angiostatin, and endostatin, thrombospondin (TSP), pigment epithelium derived factor (PEDF), transforming growth factor beta (TGF-beta), ranibizurnab (LUCENTIS®) or bevacizumab (AVASTIN®), tissue inhibitors of metalloproteinases (TiMPs), Plasminogen Activator Inhibitor (PAI), prolactin, interferons, and angiopoietin 2. Hormones and hormone antagonists may include 17B-estradiol, adrenocorticotropic hormone, adrenomedullin, alpha-melanocyte stimulating hormone, chorionic gonadotropin, corticosteroid-binding globulin, corticosterone, dexamethasone, estriol, follicle stimulating hormone, gastrin 1, glucagon, gonadotropin, hydrocortisone, insulin, insulin-like growth factor binding protein. L-3,3′,5′-triiodothyronine, L-3,3′,5′-triiodothyronine, leptin, luteinizing hormone, L-thyroxine, melatonin, MZ4, oxytocin, parathyroid hormone. PEC-60, pituitary growth hormone, progesterone, prolactin, secretin, sex hormone binding globulin, thyroid stimulating hormone, thyrotropin releasing factor, thyroxine-binding globulin, and vasopressin. Cell nutrients may include but are not limited to albumin, B-27 supplement, fetal bovine serum (FBS), ethanolamine, fetuin, glutamine, insulin, peptone, purified lipoprotein material, sodium selenite, transferrin, vitamin A, vitamin B9, vitamin B12, vitamin C, or vitamin E, glucose, galactose, riboflavin, thiamine, biotin, cholesterol, iron zinc, copper, selenium, magnesium, and tricarboxylic acid. Substrates supporting cell growth, survival, and implantation may include but are not limited to extracellular matrix from bovine corneal epithelium, Matrigel TM (basement membrane substrate), or gelatin.

Also embraced by the present disclosure are pharmaceutical packs and/or kits. Pharmaceutical packs and/or kits provided may comprise a pharmaceutical composition provided herein, a diluent, and instructions for thawing the pharmaceutical composition by diluting the pharmaceutical composition prior to administration to a subject, preferably without washing the cells prior to addition of the diluent. In some embodiments, the diluent is GS2, GS2 Plus, BSS, BSS Plus®, a dextran and HSA solution, PBS, DMEM, MEM, albumin, or any other appropriate diluent. In some embodiments, the diluent is GS2 or GS2 Plus. In some embodiments, the pharmaceutical composition is cryopreserved. In some embodiments, the instructions further comprise instructions for thawing the cryopreserved pharmaceutical composition, and optionally for combining the composition with the diluent and then administering the resultant composition to a subject. In some embodiments, the pharmaceutical composition is not cryopreserved. In some embodiments, the instructions further comprise instructions for combining the composition with the diluent and then administering the resultant composition to a subject.

Some aspects of this disclosure provide methods that comprise contacting a population of cells with a cell cryopreservation formulation provided herein. The population of cells may be a population of RPE cells, a population of mesenchymal stem cells, a population of mesenchymal cells, a population of photoreceptor cells, a population of photoreceptor progenitor cells, a population of photoreceptor rescue cells, a population of retinal ganglion cells, a population of retinal ganglion progenitor cells, a population of corneal endothelial cells, or a population of corneal endothelial progenitor cells. The population of cells may be generated by in vitro differentiation of pluripotent stem cells such as embryonic stem cells or induced pluripotent stem cells. The population of cells may be human cells or non-human cells.

Some aspects of this disclosure provide methods comprising contacting a population of cells with a cell cryopreservation formulation provided herein to form a pharmaceutical composition, and then cryopreserving the pharmaceutical composition. The pharmaceutical compositions may be cryopreserved using any suitable process, including at any appropriate temperature. For example, the pharmaceutical compositions may be cryopreserved at temperatures between −100° C. and −200° C. In some embodiments the pharmaceutical compositions are cryopreserved at temperatures below −125° C. In some embodiments the pharmaceutical compositions are cryopreserved at temperatures at or below 135° C. In some embodiments the pharmaceutical compositions are cryopreserved at temperatures below −150° C. The pharmaceutical compositions may be cryopreserved in liquid nitrogen or in the gas phase of liquid nitrogen.

Some aspects of this disclosure provide methods for treating a disease or condition comprising (a) thawing a cryopreserved cell preparation provided herein by diluting the cell preparation with a diluent at a ratio in the range of about 1:1 to about 1:20, or about 1:2 to about 1:20, or about 1:3 to about 1:20, or about 1:4 to about 1:20, or about 1:5 to about 1:20, or about 1:4 to about 1:19, or about 1:4 to about 1:18, or about 1:4 to about 1:17, for example, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19 and 1:20 or more, for example 1:40, 1:50, 1:60, 1:70, 1:80, 1:90 or 1:100 to form a diluted cell preparation, and (b) administering the diluted cell preparation to the subject. In some embodiments, the diluent is GS2 or GS2 Plus. In some embodiments, the cell preparation is diluted with warmed diluent. In some embodiments, the cell preparation is diluted with cold diluent. In some embodiments, the cell preparation is pipetted gently after dilution. In some embodiments, the cell preparation is pipetted vigorously after dilution. As provided in the examples, there were no differences between embodiments in which the cell preparation was diluted with a warmed diluent and embodiments in which the cell preparation was diluted with cold diluent. There were also no differences in the viability for cells between embodiments in which the cell preparations were pipetted gently after dilution and embodiments in which the cell preparations were pipetted vigorously after dilution, for example into the storage container or any container into which the cell preparations are transferred to for dilution.

### Exemplary Uses and Methods

The cell preparations disclosed herein, including the cryopreserved formulations once thawed, can be used in various clinical applications. As noted herein, the cryopreserved formulations may be thawed through simple addition of a diluent such as GS2 or GS2 Plus, following which they may be administered to a subject without the need for wash or other manipulation. In some embodiments, the cryopreserved formulations may be thawed without addition of a diluent, following which they may be administered to a subject without the need for wash or other manipulation.

The cryopreserved formulations may comprise dissociated cells or cell monolayers or cellular aggregates or clusters or sheets. The cryopreservative formulations provided herein are compatible with various cell types, cell populations, and tissues, including, but not limited to, adult stem and progenitor cells, differentiated cells, and populations and tissues comprising such cells. In some embodiments, the cell, cell population, or tissue so formulated is intended for clinical use in a regenerative medicine approach. For example, the cell, cell population, or tissue may be a cell, a cell population, or a tissue that can replace cells lost or degenerated in a subject, or repair or replace a tissue that has been damaged or is dysfunctional in a subject. For example, in some embodiments, the cell, cell population, or tissue may comprise a pluripotent stem cell, for example an iPS cell or an ES cell, a multipotent stem or progenitor cell, or a functional differentiated cell, or a population or tissue comprising such cells or a combination of such cells.

The cells, cell populations, or tissues to be cryopreserved using the formulations and methods disclosed herein include but are not limited to an RPE cell, a photoreceptor cell, a photoreceptor progenitor cell, a photoreceptor rescue cell, a corneal endothelial cell, a corneal endothelial progenitor cell, a mesenchymal cell, a mesenchymal stem cell, a hematopoietic stem cell, a neural or neuronal stem or progenitor cell, a glial stem or progenitor cell, a pancreatic stem or progenitor cell, a beta cell, a keratinocyte, a chondrocyte, an osteoblast, an osteoclast, or a population or tissue comprising or consisting essentially of such cells, e.g., a monolayer or sheet or aggregate or cluster of RPE cells, corneal endothelial cells, photoreceptor rescue cells, a pancreatic islet, or a skin graft.

In some embodiments, the cryopreserved cell preparation can be stored indefinitely or it can be stored for defined periods of time, and optionally it can be transported to a clinic or other end user location. Significantly, it can be thawed through simple addition of a diluent, and then administered to a subject without further manipulation. The cryopreserved formulation is typically a low volume formulation. For example, it may have a volume of less than 1 mL, less than 500 μL, less than 250 μL, less than 100 μL, or less than or about 50 μL or any volume disclosed herein. It may be diluted at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, at least about 20-fold, at least about 25-fold, at least about 30-fold, at least about 35-fold, at least about 40-fold, at least about 45-fold, at least about 50-fold, at least about 60-fold, at least about 70-fold, at least about 80-fold, at least about 90-fold, at least about 100-fold, or more, in order to arrive at a volume and cell concentration suitable for the intended clinical use. The ratio of cryoprotective formulation volume to diluent volume may be expressed as a ratio and may include but is not limited to ratios of about 1:1 to about 1:20, or about 1:2 to about 1:20, or about 1:3 to about 1:20, or about 1:4 to about 1:20, or about 1:5 to about 1:20, or about 1:4 to about 1:19, or about 1:4 to about 1:18, or about 1:4 to about 1:17, for example, 1:1, 1:2, 1:3, 1:4, 1:5, 1:6, 1:7, 1:8, 1:9. 1:10, 1:11, 1:12, 1:13, 1:14, 1:15, 1:16, 1:17, 1:18, 1:19, 1:20, 1:21, 1:22, 1:23, 1:24, 1:25, 1:30, 1:35, 1:40, 1:45, 1:50, 1:55, 1:60, 1:65, 1:70, 1:75, 1:80, 1:85, 1:90, 1:95, 1:100 or more.

The cryopreserved cell preparation typically comprises cells at a high density such as but not limited to at least or about 3×10² per uL, or at least or about 10³ per μL, at least or about 2×10³ per μL, at least or about 3×10³ per μL, at least or about 3.5×10³ per μL, at least or about 5×10³ per μL, at least or about 10⁴ per μL, at least or about 5×10⁴ per μL, at least or about 10⁵ per μL, or at least or about 5×10⁵ per μL, or higher. As will be understood by the skilled person, the cell concentration and the intended use will guide the degree of dilution that is required prior to administration to a subject.

The cryopreserved formulations may be stored in a frozen state, such as in the vapor phase of a liquid nitrogen tank, for an extended period of time, such as those recited herein and including but not limited to at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, at least 1 year, at least 2 years, at least 3 years, at least 4 years at least 5 years or more with minimal impact on cell viability or cellular function, as measured for example by dye exclusion, re-plating efficiency, or repopulating capacity. Cells or cell populations cryopreserved and thawed according to the present disclosure may have a cell viability, re-plating efficiency, and/or repopulation capacity of at least 30%, at least 35%, at least 40%, at least 45%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% of the viability, re-plating efficiency, and/or repopulation capacity prior to cryopreservation.

In some embodiments, the cryopreserved cell preparations provided herein are stored at temperatures between −100° C. and −200° C., including at temperatures below −125° C., at temperatures below −135° C., or at temperatures below −150° C. In some embodiments, the cryopreserved cell preparations provided herein are stored in liquid nitrogen or in the vapor phase of liquid nitrogen.

The cryopreserved cell preparations are produced by combining the cells, cell population, or tissue of interest in the cryoprotective formulation provided herein, optionally dispersing the cells, cell population, tissue in the formulation, and then slow-freezing the mixture. Slow-freezing can be accomplished manually or by device, as described in the Examples. In one embodiment, the preparation is frozen at a rate of −1° C. per minute until a desired, optionally intermediate, temperature is reached, such as for example −80° C. Alternatively, the rate of freezing may be −1.5° C. per minute or −2° C. per minute, etc. The cryopreserved cell preparation may be kept at the intermediate temperature for hours, days, or weeks, or longer. It may then be kept at a lower temperature. As an example, the preparation may be brought to −80° C. using a slow-freeze process, after which it may be stored at −80° C. overnight (or for up to about 12 or about 24 hours), after which it may be stored in the vapor phase of a liquid nitrogen tank (e.g., at about −125° C.). Once at the lower temperature, it may be stored there for days, weeks, months, or even years. It may even be transported, for example to a clinical site, while in the cryopreserved form at the lower temperature, if so desired. The temperature of the cryoprotective formulation can, for example, between 0° C. and 45° C., for example, about 1° C., about 2° C., about 3° C., about 4° C., about 10° C., about 15° C., about 20° C., about 25° C., about 30° C., about 31° C., about 32° C., about 33° C., about 34° C., about 35° C., about 36%, about 37° C. and about 40° C.

Some aspects of this disclosure provide methods for treating a subject having a disorder or condition that would benefit from a cell transplant. The methods may include thawing a cryopreserved cell preparation comprising a cryopreservative formulation as provided herein, and then administering the thawed cell preparation to a subject in need thereof, optionally without dilution with a diluent. In some embodiments, the methods may include thawing a cryopreserved cell preparation comprising a cryopreservative formulation as provided herein, by addition of a diluent, and then administering the thawed and diluted cell preparation to a subject in need thereof. In some embodiments, the diluent is GS2, GS2 Plus, BSS, BSS Plus®, a dextran and HSA solution, PBS, DMEM, MEM, or albumin. In some embodiments, the diluent is GS2 or GS2 Plus. In some embodiments, the diluent is GS2 or GS2 Plus. The subject may have or may have been diagnosed with a disease or disorder that can be treated by administering a cell, cell population, or tissue. The cell preparation is administered in a cell dose effective to treat the disorder or condition in the subject. In some embodiments, the disorder or condition includes, but is not limited to, retinal detachment, retinal dysplasia, Angioid streaks, Myopic Macular Degeneration, or retinal atrophy or associated with a number of vision-altering ailments that result in photoreceptor damage and blindness, such as, for example, choroideremia, diabetic retinopathy, macular degeneration (e.g., age-related macular degeneration), dry or wet age-related macular degeneration, retinitis pigmentosa, and Stargardt's Disease (fundus flavimaculatus). In some embodiments, the method further comprises monitoring at least one symptom of the disorder or condition in the subject, optionally in order to determine if such symptom is lessened in severity or frequency post-administration.

The terms “treatment,” “treat,” and “treating” refer to a clinical intervention aimed to reverse, alleviate, delay the onset of, or inhibit the progress of a disorder or condition. In some instances, the effect of the intervention may be assessed by monitoring symptoms associated with the disorder or condition. In some embodiments, the cell preparation may be administered after one or more symptoms have developed and/or after a disorder or condition has been diagnosed. In other embodiments, the cell preparation may be administered in the absence of symptoms, e.g., to prevent or delay onset of a symptom or inhibit onset or progression of a disorder or condition. For example, the cell preparation may be administered to a susceptible individual prior to the onset of symptoms (e.g., in light of a history of symptoms and/or in light of genetic or other susceptibility factors). The cell preparation may be administered once or more than once, including at regular and defined time points or on an as-needed basis.

The subject may be a human or a non-human mammal. In some embodiments, the subject is a non-human primate. In some embodiments, the subject is a rodent. In some embodiments, the subject is a sheep, a goat, a cattle, a cat, or a dog. In some embodiments, the subject is a research animal. In some embodiments, the subject is genetically engineered, e.g., a genetically engineered non-human subject. The subject may be of either sex and at any stage of development.

The term “effective amount,” as used herein, refers to an amount of a biologically active agent that is sufficient to elicit a desired biological response. For example, in some embodiments, an effective amount of a cell preparation may refer to the amount of the preparation that comprises a number of cells or amount of tissue that is sufficient to provide a therapeutic benefit to a subject having a disorder or condition, e.g., sufficient to improve vision in a subject with a retinal disease or disorder. As will be appreciated by the skilled artisan, the effective amount may vary depending on various factors as, for example, on the desired biological response, e.g., on the specific disorder being treated, a specific symptom to be alleviated, or the cell or tissue being targeted, and on the subject's age, gender, and general health status.

Some aspects of this disclosure provide methods for treating a retinal disease, wherein the methods comprise administering an effective amount of a cell preparation provided herein to the eye of a subject having a retinal disease. In some embodiments, the subject has or is diagnosed with the retinal disease. In some embodiments, the retinal disease is rod or cone dystrophy, retinal degeneration, retinitis pigmentosa, diabetic retinopathy, macular degeneration, for example wet or dry age-related macular degeneration, Leber congenital amaurosis, or Stargardt disease. In some embodiments, the cells being administered comprise RPE cells, photoreceptor cells, photoreceptor progenitor cells, photoreceptor rescue cells, corneal endothelial cells, corneal endothelial progenitor cells, or retinal ganglion cells or retinal ganglion progenitor cells, any of which may be generated by in vitro differentiation of pluripotent stem cells such as embryonic stem cells or induced pluripotent stem cells.

### Kits

Some aspects of this disclosure provide kits comprising (a) a cryopreserved pharmaceutical composition comprising cells of interest as provided herein; (b) a diluent such as but not limited to GS2 or GS2 Plus, and (c) instructions for adding the diluent to the composition, thereby thawing and diluting the composition, and rendering it suitable for administration to a subject. Those instructions may also provide that no washing or other manipulation of the cryopreserved composition is necessary post-thaw. The kit may further comprise a device for administering the composition such as a syringe and needle.

This disclosure therefore provides inter alia the following:

Clause 1. A formulation comprising about 4-10% (v/v) cryoprotectant, about 2-8% (w/v) albumin, about 0-1.5% (w/v) glucose, and a buffer.

Clause 2. The formulation of clause 1, wherein the cryoprotectant is selected from DMSO, glycerol, and ethylene glycol.

Clause 3. The formulation of clause 1, wherein the cryoprotectant is DMSO.

Clause 4. The formulation of any one of the foregoing clauses, wherein the formulation comprises about 4-6% (v/v) cryoprotectant.

Clause 5. The formulation of any one of the foregoing clauses, wherein the formulation comprises about 0.08-0.10% (w/v) glucose.

Clause 6. The formulation of any one of the foregoing clauses, wherein the formulation comprises about 5% (v/v) DMSO, about 2.5% (w/v) albumin, about 0.09% (w/v) glucose, and a buffer.

Clause 7. The formulation of any one of clauses 1-4, wherein the formulation comprises about 0.6% (w/v) glucose.

Clause 8. The formulation of any one of clauses 1-4 and 7, wherein the formulation comprises about 5% DMSO, about 2.5% albumin, about 0.6% glucose, and a buffer.

Clause 9. The formulation of any one of the foregoing clauses, wherein albumin is human albumin.

Clause 10. The formulation of any one of the foregoing clauses, wherein albumin is recombinant human albumin.

Clause 11. The formulation of any one of the foregoing clauses, wherein the buffer is buffered saline.

Clause 12. The formulation of any one of the foregoing clauses, wherein the buffer is phosphate-buffered saline (PBS).

Clause 13. The formulation of clause 11 or 12, wherein the buffered saline comprises Ca2+ and Mg2+.

Clause 14. The formulation of clause 11 or 12, wherein the buffered saline does not comprise Ca2+ and Mg2+.

Clause 15. The formulation of any one of the foregoing clauses, further comprising a cell population or a tissue.

Clause 16. The formulation of clause 15, wherein the cell population is a dissociated cell population.

Clause 17. The formulation of clause 15, wherein the cell population comprises a cell monolayer, a cell sheet, cellular clusters, cellular spheres or cellular aggregates.

Clause 18. The formulation of clause 15, wherein the cell population comprises retinal pigment epithelium (RPE) cells, photoreceptor rescue cells, photoreceptor progenitor cells, or corneal endothelial cells.

Clause 19. The formulation of any one of the foregoing clauses, wherein the formulation is cryopreserved.

Clause 20. The formulation of any one of the foregoing clauses, wherein the formulation does not comprise a polymeric excipient.

Clause 21. The formulation of any one of the foregoing clauses, wherein the formulation does not comprise dextran.

Clause 22. A cell preparation comprising
(a) the formulation of any one of clauses 1-14 and
(b) a population of cells.

Clause 23. The cell preparation of clause 22, wherein the population of cells comprises retinal pigment epithelium (RPE) cells, photoreceptor rescue cells, photoreceptor progenitor cells or corneal endothelial cells.

Clause 24. The cell preparation of clause 22 or 23, wherein the population of cells is at a concentration of about 10,000 cells-100,000 cells/μL in the cell preparation.

Clause 25. The cell preparation of clause 22 or 23, wherein the population of cells is at a concentration of about 15,000-50,000 cells/μL in the cell preparation.

Clause 26. The cell preparation of any one of clauses 22-25, in a cryopreservation vial, syringe, ampoule, bottle or cooler package.

Clause 27. The cell preparation of clause 26, wherein about 10 to about 500 μL of the preparation are in the vial, syringe, ampoule, bottle or cooler package.

Clause 28. The cell preparation of clause 26 or 27, wherein about 10⁵ to 10⁷ cells are present in the vial, syringe, ampoule, bottle or cooler package.

Clause 29. The cell preparation of clause 24 or 25, wherein about 10⁵ to 5×10⁶ cells are present in the vial, syringe, ampoule, bottle or cooler package.

Clause 30. The cell preparation of clause 24 or 25, wherein about 10⁵ to 2×10⁶ cells are present in the vial, syringe, ampoule, bottle or cooler package.

Clause 31. The cell preparation of any one of clauses 22-30, wherein the cell preparation is cryopreserved.

Clause 32. A method of preparing a cell preparation, comprising contacting a formulation of any one of clauses 1-14 with a population of cells.

Clause 33. The method of clause 29, further comprising cryopreserving the cell preparation.

Clause 34. The method of clause 32 or 33, wherein the population of cells comprises retinal pigment epithelium (RPE) cells, photoreceptor rescue cells, photoreceptor progenitor cells or corneal endothelial cells.

Clause 35. A method of treating a subject having a disorder or condition comprising, thawing the cell preparation of clause 31, diluting the cell preparation with a diluent at a ratio selected from the group consisting of 1:2, 1:3, 1:4 and 1:5, and administering the diluted cell preparation to the subject.

Clause 36. The method of clause 35, wherein the disorder or condition is selected from the group consisting of retinal detachment, retinal dysplasia, Angioid streaks, Myopic Macular Degeneration, or retinal atrophy or associated with a number of vision-altering ailments that result in photoreceptor damage and blindness, such as, for example, choroideremia, diabetic retinopathy, macular degeneration (e.g., age related macular degeneration), retinitis pigmentosa, and Stargardt's Disease (fundus flavimaculatus).

Clause 37. The method of clause 35, wherein the diluent is GS2, GS2 Plus, BSS, BSS Plus®, a dextran and HSA solution, PBS, DMEM, MEM, or albumin.

Clause 38. A method of treating a subject having a disorder or condition comprising, thawing the cell preparation of clause 31, diluting the cell preparation with a diluent at a ratio selected from the group consisting of 1:15, 1:16, 1:17, 1:18 and 1:19, and administering the diluted cell preparation to the subject.

Clause 39. The method of clause 37, wherein the disorder or condition is selected from the group consisting of retinal detachment, retinal dysplasia, Angioid streaks, Myopic Macular Degeneration, or retinal atrophy or associated with a number of vision-altering ailments that result in photoreceptor damage and blindness, such as, for example, choroideremia, diabetic retinopathy, macular degeneration (e.g., age related macular degeneration), retinitis pigmentosa, and Stargardt's Disease (fundus flavimaculatus).

Clause 40. The method of clause 38, wherein the diluent is GS2 or GS2 Plus, BSS, BSS Plus®, a dextran and HSA solution, PBS, DMEM, MEM, or albumin.

Clause 41. A kit comprising
(a) the cryopreserved cell preparation of clause 31,
(b) a diluent, and
(c) instructions for diluting the cell preparation prior to administration to a subject.

Clause 42. The kit of clause 41, wherein the instructions further comprise instructions for thawing the cryopreserved cell preparation.

Clause 43. A pharmaceutical composition comprising the formulation of any one of clauses 1-14 in combination with a population of cells.

Clause 44. The pharmaceutical composition of clause 43, wherein the population of cells comprises retinal pigment epithelium (RPE) cells, photoreceptor rescue cells, photoreceptor progenitor cells or corneal endothelial cells.

Clause 45. The pharmaceutical composition of clause 43, wherein the population of cells comprises neural cells, neural stem cells, or neural precursor cells.

Clause 46. A pharmaceutical composition comprising the formulation of any one of clauses 15-21 and a diluent, wherein the formulation to diluent ratio is in the range of 1:2 to 1:20, wherein the pharmaceutical composition is suitable for administration to a subject, optionally administration to the eye, further optionally administration to the subretina.

Clause 47. A pharmaceutical composition comprising the cell preparation of any one of clauses 22-25 and 31 and a diluent, wherein the cell preparation to diluent ratio is in the range of 1:2 to 1:20, wherein the pharmaceutical composition is suitable for administration to a subject, optionally administration to the eye, further optionally administration to the subretina.

Clause 48. A cell preparation comprising the formulation of any one of clauses 1-14, 19, 20 and 21 and a diluent, wherein the formulation to diluent ratio is in the range of 1:2 to 1:20, wherein the formulation comprises a cell population, wherein the cell preparation is suitable for administration to a subject, optionally administration to the eye, further optionally administration to the subretina.

Clause 49. The pharmaceutical composition of clause 46 or 47 or the cell preparation of clause 48, wherein the pharmaceutical composition or the cell preparation is not washed prior to or after dilution with the diluent.

Clause 50. The pharmaceutical composition of clause 46 or 47 or the cell preparation of clause 48, wherein the pharmaceutical composition or the cell preparation is not centrifuged prior to or after dilution with the diluent.

Clause 51. The pharmaceutical composition of clause 46 or 47 or the cell preparation of clause 48, wherein the formulation or the cell preparation is cryopreserved prior to dilution with the diluent.

Clause 52. The pharmaceutical composition or the cell preparation of any one of clauses 48-51, wherein DMSO is present in detectable amounts at a level that is equal to or less than about 1% (v/v).

Clause 53. The pharmaceutical composition or the cell preparation of any one of clauses 48-51, wherein DMSO is present in detectable amounts at a level that is equal to or less than about 0.037 mg/dose.

Clause 54. A cell preparation comprising the formulation of any one of clauses 1-14 and a population of cells, wherein the cell preparation is thawed by dilution with a diluent and is unwashed.

Clause 55. The cell preparation of clause 54, wherein the cell preparation is diluted with a diluent at a ratio selected from the group consisting of 1:2 to 1:20.

Clause 56. The cell preparation of clause 54 or 55, wherein DMSO is present in detectable amounts at a level that is equal to or less than about 0.037 mg/dose.

Clause 57. The cell preparation of any one of clauses 54-56, wherein the diluent is GS2 or GS2 Plus.

### EXAMPLES

The presently disclosed subject matter now being generally described, it will be more readily understood by reference to the following examples, which are included merely for purposes of illustration of certain aspects and embodiments of the presently disclosed subject matter, and are not intended to limit the presently disclosed subject matter.

### Example 1—Cryoprotective Formulations Preserve Post-Thaw Cell Viability and Cell Growth

RPEs derived from human embryonic stem cells were pooled in a 225 mL bottle and washed with 200 mL of 0.5% rHA/DPBS by centrifugation at 160× g for 5 minutes. Washing was repeated 3 times in total.

RPE cells were resuspended with 0.09% Glucose/2.5% rHA/DPBS, followed by mixing with buffers containing DMSO, rHA and glucose at the volume ratio of 1:1. Nine (9) formulations containing different concentrations of DMSO, albumin and glucose were prepared as shown in Table 1.

TABLE 1

| Components | MB#1 | DMSO-H | DMSO-L | Alb-H | Alb-L | Glu-H | Glu-L | HHH | LLL |
|---|---|---|---|---|---|---|---|---|---|
| RPE Cell | 50,000 cells/uL | | | | | | | | |
| DMSO (v/v) | 5% | 6% | 4% | 5% | 5% | 5% | 5% | 6% | 4% |
| Recombinant Albumin (w/v) | 2.5% | 2.5% | 2.5% | 3.0% | 2.0% | 2.5% | 2.5% | 3.0% | 2.0% |
| Glucose (w/v) | 0.09% | 0.09% | 0.09% | 0.09% | 0.09% | 0.10% | 0.08% | 0.10% | 0.08% |

| | |
|---|---|
| DPBS w/Ca, Mg | q.s. |

Thirty (30) μL of formulated RPE cells were filled in Crystal Zenith® (CZ) vials (West Pharmaceutical Services, Inc. Exton, PA) and cryopreserved using a Corning® CoolCell® container (Corning Life Sciences, Tewksbury, MA) for slow cryopreservation. The vials were then transferred and stored in the vapor phase of liquid nitrogen.

Pre-freeze cell viability was evaluated using the Trypan Blue cell counting method. As a result, no significant difference was observed among the nine formulations (see FIG. 1). Pre-freeze viability was higher than 95% with all formulations.

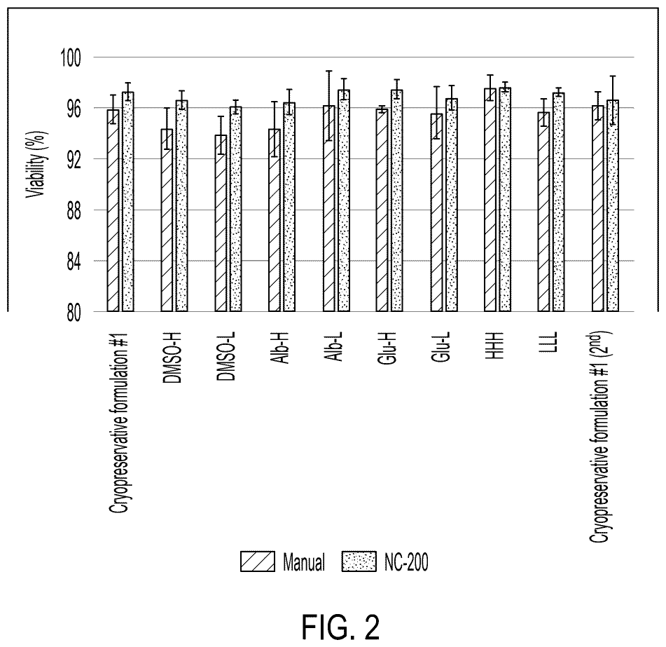

Post-thaw cell viability was evaluated using a manual Trypan Blue cell counting method and an automated method using the NucleoCounter® NC-200™ (ChemoMetec Inc. Bohemia, NY) device. No significant differences were observed among the nine formulations using either method (see FIG. 2) (n=3). Post-thaw viability was higher than 90% with all formulations and ranged from 94% to 98%.

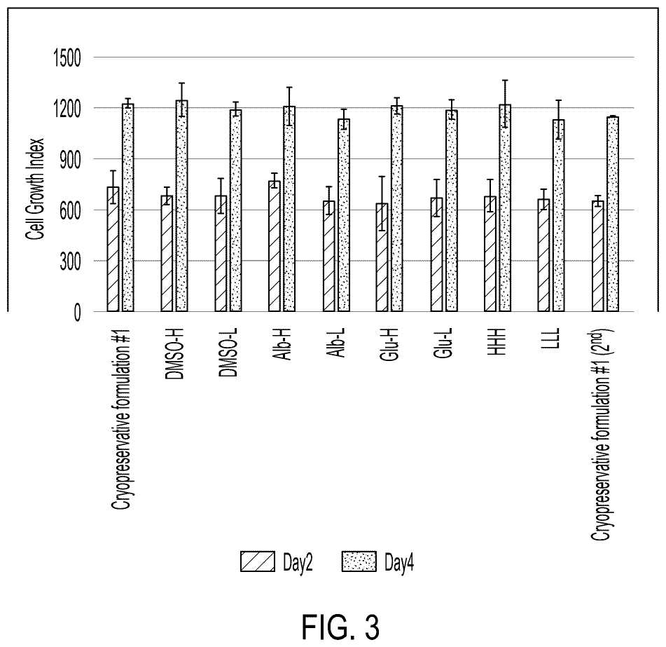

Post-thaw cell growth was evaluated using the CyQUANT® Cell Proliferation Assay (Thermo Fisher Scientific, Waltham, MA). Cryopreserved RPE vials were thawed, seeded and cultured in growth medium in 96 well plates. Plates were frozen down at day 2 and day 4 to evaluate their cell growth index which is defined by the DNA concentration extracted from cells following cell lysis. No significant difference was observed among the nine formulations (see FIG. 3).

### Example 2—Cryoprotectants Reduce Generation of Multinucleated Cells

RPE cells derived from human embryonic stem cells were pooled in a 50 mL tube and washed with 30 mL of 0.5% rHA/DPBS by centrifugation at 160× g for 5 minutes. Washing was repeated 3 times in total.

RPE cells were resuspended at a density of 50K cell/μl in 2.5% rHA/DPBS, followed by mixing with cryoprotectant-containing buffer at a volume ratio of 1:1. Five (5) formulations containing 1% DMSO, 2.5% DMSO, 5% DMSO, 5% Glycerol or 5% Ethylene Glycol, each of the 5 formulations containing 50 mM Trehalose, 7.5% Dextran 40 and 2.5% rHA in DPBS were prepared as shown in Table 2.

TABLE 2

| | A | B | C | D | E |
|---|---|---|---|---|---|
| DMSO | 1% | 2.5% | 5% | — | — |
| Glycerol | | | | 5% | — |
| Ethylene Glycol | — | — | — | — | 5% |

| | |
|---|---|
| Excipients | 50 mM Trehalose, 7.5% Dextran 40, |
| | 2.5% rAlbumin, PBS |
| Cell Density | 50K cells/μL |

Ten (10) μL of formulated RPE cells were added to cryovials and cryopreserved using Mr.Frosty™ Freezing Container (Thermo Fisher Scientific, Inc., Waltham, MA) for slow cryopreservation. Vials were then transferred and stored in the vapor phase of liquid nitrogen.

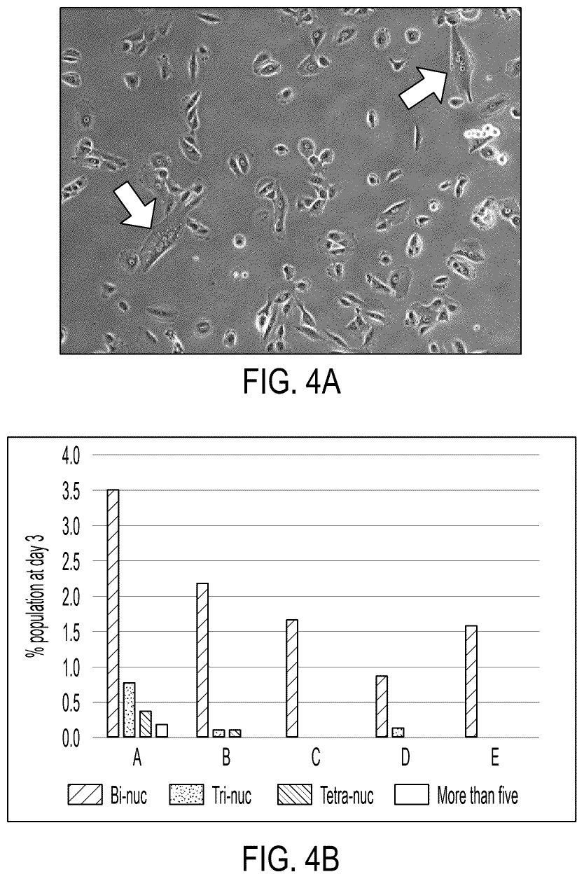

Cryopreserved RPE cells were thawed, seeded and cultured in growth medium for 3 days, and evaluated for the presence of multinucleated cells under a microscope. RPE cells in Formulations A (1% DMSO) showed the presence of some multinucleated cells which had more than four nuclei in a cell in a field of several hundred RPE cells (see FIG. 4A), although Formulation B (2.5% DMSO), showed one multinucleated cell, with more than four nuclei in a cell in a field of several hundred RPE cells.

RPE cells in Formulations C (5% DMSO) and E (5% Ethylene Glycol), showed no multinucleated cells having more than three nuclei in a cell in a field of among several hundred RPE cells, and RPE cells in Formulation D (5% Glycerol) did not show any multinucleated cells having more than four nuclei in a cell in a field of among several hundred RPE cells (FIG. 4B).

These results indicate that cell penetrating cryoprotectants such as DMSO, Glycerol, and Ethylene Glycol reduce the generation of multinucleated cells.

### Example 3—Effect of Other Excipients on Multinucleation, Cell Growth, and Viability

RPE cells derived from human embryonic stem cells were pooled in a 50 mL tube and washed with 30 mL of 0.5% rHA/DPBS by centrifugation at 160x g for 5 minutes. Washing was repeated 3 times in total.

RPE cells were resuspended with 2.5% rHA/DPBS, followed by mixing with cryoprotectant-containing buffer at a volume ratio of 1:1. Eight (8) formulations containing varying amounts of glycerol and Dextran 40, each containing 0.09% glucose and 2.5% rHA in DPBS (with or without Ca, Mg) were prepared as shown in Table 3.

TABLE 3

| Condition | Excipient 1 | Excipient 2 | Others | Cell Density |
|---|---|---|---|---|
| G5D10 PBS− | 5% Glycerol | 10% Dextran 40 | 0.09% Glucose | 50K cells/μL |
| G2.5D10 PBS− | 2.5% Glycerol | 10% Dextran 40 | 2.5% rAlbumin | |
| G5D7.5 PBS− | 5% Glycerol | 7.5% Dextran 40 | PBS (−Ca, −Mg) | |
| G2.5D7.5 PBS− | 2.5% Glycerol | 7.5% Dextran 40 | | |
| G5D10 PBS+ | 5% Glycerol | 10% Dextran 40 | 0.09% Glucose | |
| G2.5D10 PBS+ | 2.5% Glycerol | 10% Dextran 40 | 2.5% rAlbumin | |
| G5D7.5 PBS+ | 5% Glycerol | 7.5% Dextran 40 | PBS (+Ca, +Mg) | |
| G2.5D7.5 PBS+ | 2.5% Glycerol | 7.5% Dextran 40 | | |

Ten (10) μL of formulated RPE cells were added to cryovials and cryopreserved using Mr.Frosty™ Freezing Container (Thermo Fisher Scientific, Inc., Waltham, MA) for slow cryopreservation. Vials were then transferred and stored in the vapor phase of liquid nitrogen.

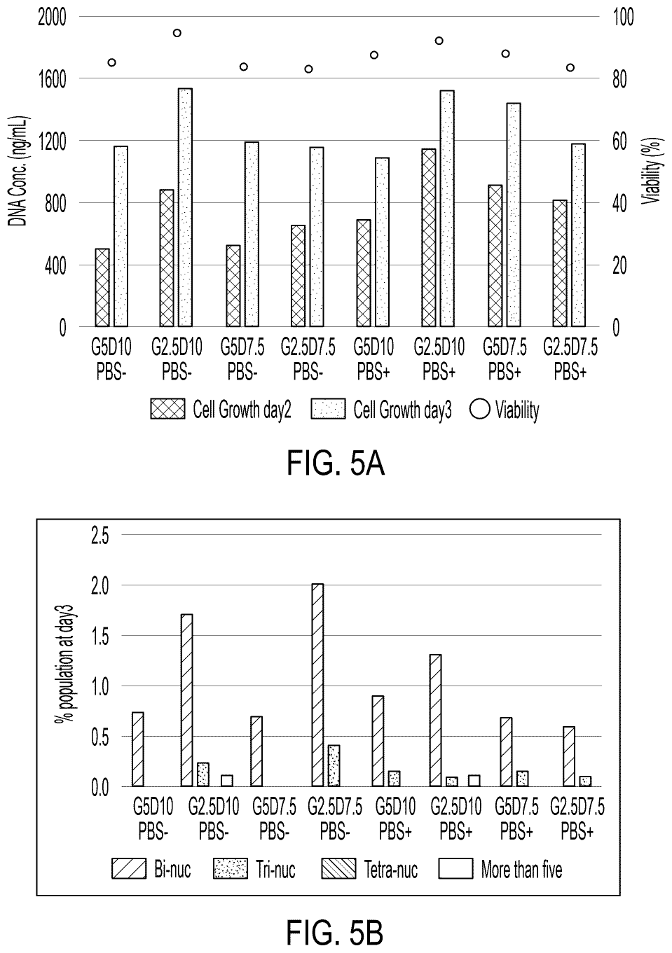

Post-thaw cell viability was evaluated using the Trypan Blue cell counting method. Post-thaw cell growth was also evaluated using the CyQUANT® Cell Proliferation Assay (Thermo Fisher Scientific, Waltham, MA). Cryopreserved RPE vials were thawed, seeded and cultured in growth medium in 96 well plates. Plates were frozen down at day 2 and day 3 to evaluate DNA concentration as a cell growth index. No significant difference was observed among the eight formulations (see FIG. 5A) (viability: n=1, cell growth: n=3). Multinucleated cells were observed with all 2.5% Glycerol conditions, but no multinucleated cells were observed with 5% Glycerol (FIG. 5B).

RPE cells were pooled in a 50 mL tube and washed with 30 mL of 0.5% rHA/DPBS by centrifugation at 160x g for 5 minutes. Washing was repeated 3 times in total.

RPE cells were resuspended with 2.5% rHA/DPBS, followed by mixing with cryoprotectant-containing buffer at a volume ratio of 1:1. Five (5) formulations were prepared as shown in Table 4.

TABLE 4

| Condition | Excipient 1 | Excipient 2 | Others | Cell Density |
|---|---|---|---|---|
| D5 | 5% DMSO | — | 0.09% Glucose | 50K cells/μL |
| G5 | 5% Glycerol | 7.5% Dextran 40 | 2.5% rAlbumin | |
| D0 | — | 7.5% Dextran 40 | PBS (−Ca, −Mg) | |
| G2.5 | 2.5% Glycerol | 7.5% Dextran 40 | | |
| P2.5 | 2.5% Propanediol | 7.5% Dextran 40 | | |

Ten (10) μL of formulated RPE cells were added to cryovials and cryopreserved using Mr. Frosty™ Freezing Container (Thermo Fisher Scientific, Inc., Waltham, MA) for slow cryopreservation. Vials were then transferred and stored in the vapor phase of liquid nitrogen.

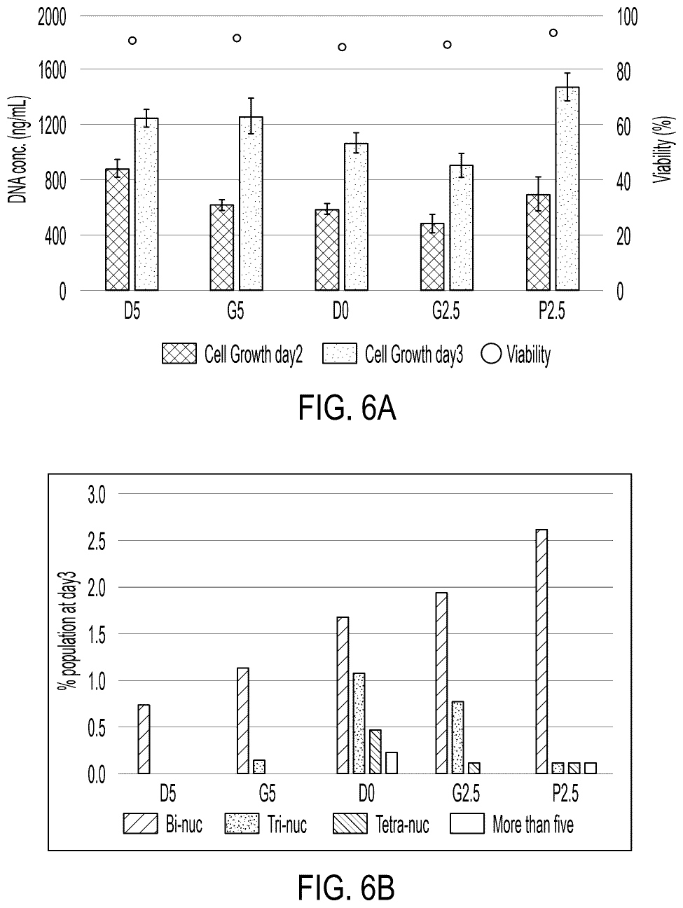

Post-thaw cell viability was evaluated using the trypan blue cell counting method. Post-thaw cell growth was also evaluated using the CyQUANT® Cell Proliferation Assay (Thermo Fisher Scientific, Waltham, MA). Cryopreserved RPE vials were thawed, seeded and cultured in growth medium in 96 well plates. Plates were frozen down at day 2 and day 3 to evaluate DNA concentration as a cell growth index. No significant difference was observed among the five formulations (see FIG. 6A) (viability: n=1, cell growth: n=3). Multinucleated cells were observed with 2.5% glycerol and 2.5% propanediol (e.g., 1,2 propanediol) but no multinucleated cells were observed with 5% DMSO or 5% glycerol (see FIG. 6B).

### Example 4—Albumin Effect on Adsorption and Cryoprotection

RPE cells were pooled in a 225 mL bottle and washed with 200 mL of 0.5% rHA/DPBS by centrifugation at 500× g for 5 minutes. Washing was repeated 4 times in total.

RPE cells were resuspended with 2.5% rHA/0.09%Glucose/DPBS, followed by mixing with DMSO-containing buffer at a volume ratio of 1:1. Four (9) formulations containing cells at different densities and albumin concentrations were prepared as shown in Table 5.

TABLE 5

| Cell Density | DMSO | rhAlbumin | Glucose | Buffer |
|---|---|---|---|---|
| 50,000 | 5% | 2.5% | 0.09% | DPBS |
| cells/μL | | — | | |
| 15,000 | | 2.5% | | |
| cells/μL | | — | | |

Thirty (30) μL of formulated RPE cells were added to Crystal Zenith® (CZ) vials (West Pharmaceutical Services, Inc. Exton, PA) and cryopreserved using a controlled rate freezer under different cryopreservation conditions, for example, different types of storage containers, different pre-freeze storage temperature, and different pre-freeze duration, as shown in Table 6. Vials were then transferred and stored in the vapor phase of liquid nitrogen.

z,999

Post-thaw cell viability was evaluated using the trypan blue method using Vi-CELL BLU Cell Viability Analyzer (Beckman Coulter, Inc., Indianapolis, IN). The formulations containing albumin showed higher post-thaw viability than the formulations without albumin especially when stored for up to 6 hours before freezing as shown in Table 7.

TABLE 7

| | | Freeze | Pre-freeze | | | | |
|---|---|---|---|---|---|---|---|
| | | Time | store | | TCD | VCD | Viability |
| Dose | rHSA | point | temperature | Container | (cells/uL) | (cells/uL) | (%) |
| 50K | + | 1 h | | CZ vial | 3040 | 2910 | 95.8 |
| | + | 1 h | | cryovial | 3148 | 3028 | 96.2 |
| | − | 1 h | | CZ vial | 3423 | 3155 | 92.2 |
| | − | 1 h | | cryovial | 3640 | 3303 | 90.8 |
| | + | 6 h | RT | cryovial | 3088 | 2888 | 93.5 |
| | + | 6 h | 4 C. | cryovial | 3003 | 2833 | 94.4 |
| | − | 6 h | RT | cryovial | 3493 | 2995 | 85.8 |
| | − | 6 h | 4 C. | cryovial | 3483 | 3118 | 89.4 |
| 15K | + | 1 h | | CZ vial | 1023 | 1000 | 97.6 |
| | + | 1 h | | cryovial | 1033 | 1008 | 97.5 |
| | − | 1 h | | CZ vial | 1010 | 933 | 92.4 |
| | − | 1 h | | cryovial | 1113 | 990 | 88.8 |
| | + | 6 h | RT | cryovial | 1110 | 1075 | 96.8 |
| | + | 6 h | 4 C. | cryovial | 1083 | 1050 | 96.8 |
| | − | 6 h | RT | cryovial | 1183 | 1025 | 86.7 |
| | − | 6 h | 4 C. | cryovial | 1175 | 1000 | 84.9 |

Based on the results, albumin shows a stabilizing effect in the formulation process.

### Example 5—Cell Drug Product Formulation Demonstrated No Toxicity

The following four (4) test vehicle or articles, as well as GS2 and GS2 Plus, were prepared as shown in Table 8 and subjected to the non-GLP acute-toxicity study.

TABLE 8

| Name of Test vehicle | Brief explanation |
|---|---|
| or Test Articles | of Test Articles |
| Cryopreservative | Cryopreservative formulation comprising 5% DMSO, 0.09% Glucose, |
| formulation #1 | 2.5% rhAlbumin and DPBS Diluted with GS2 Plus before use |
| Cryopreservative | Cryopreservative formulation comprising 5% Glycerol, 7.5% |
| formulation #3 | Dextran40, 0.09% Glucose, 2.5% rhAlbumin and DPBS |
| | Diluted with GS2 Plus before use |
| Cryopreservative | Cryopreservative formulation comprising 5% Propanediol, 7.5% |
| formulation #4 | Dextran40, 0.09% Glucose, 2.5% rhAlbumin and DPBS |
| | Diluted with GS2 Plus before use |
| CryoStor CS5 | Commercially available freezing media containing 5% DMSO |
| (BioLife Solutions) | Diluted with GS2 Plus before use |

Each of the 6 formulations were added to a plastic vial and stored at 2 to 8° C. or −10 to −30° C. Frozen formulations were thawed and diluted prior to administration, and administrated as a single unilateral dose via sub-retinal injection to NIH-Lyst^{bg}-^{J}Foxnl^{nu}Btk^{xid} (NIH III) immunocompromised mice. Animals were evaluated after 1- and 14-day observation periods. Assessment of toxicity was based on clinical observation, body weight and food consumption measurements, ophthalmic examination, and clinical and anatomic pathology evaluation.

No toxicity was observed in any of the formulations.

### Example 6—Applicability to Different Type of Cells

Photoreceptor rescue cells (PRC) were pooled in 50 mL tube and centrifuged using OptiPrep™ (STEMCELL Technologies, Inc., Vancouver, Canada) to remove dead cells and debris from cell suspension, followed by washing first with growth media at 300× g for 10 minutes then with 0.5% rHA/DPBS by centrifugation at 300× g for 10 minutes.

Photoreceptor rescue cells were resuspended with 0.5% rHA/DPBS, followed by mixing with cryoprotectant-containing buffer at a volume ratio of 1:2. PRC cells were prepared in a formulation containing 2.5% rHA, DPBS with Ca/Mg, 0.6% glucose, and different concentrations of DMSO (10% DMSO and 5% DMSO). For comparison, PRC cells were also formulated in a commercially available CryoStor® CS10 cell freezing medium (STEMCELL Technologies, Inc., Vancouver, Canada), which contains 10% DMSO.

Cells were added to Crystal Zenith® (CZ) vials (West Pharmaceutical Services, Inc. Exton, PA) at 70,000 cells/μl in 50 μL (a total of 3.5 million cells per vial) and cryopreserved at −1° C./min using a controlled rate freezer. Vials were then transferred and stored in the vapor phase of liquid nitrogen. By using 70,000 cells/μl in 50 μL the following target doses can be achieved: 1,000,000 cells in 150 μl when the cells are diluted 1:4 in buffer (for example GS2 Plus buffer (accounting for approximately 50% loss of cells due to cell death and recovery loss); 300,000 cells in 150 μl when the cells are diluted 1:17 in GS2 Plus buffer (accounting for approximately 50% loss of cells due to cell death and recovery loss).

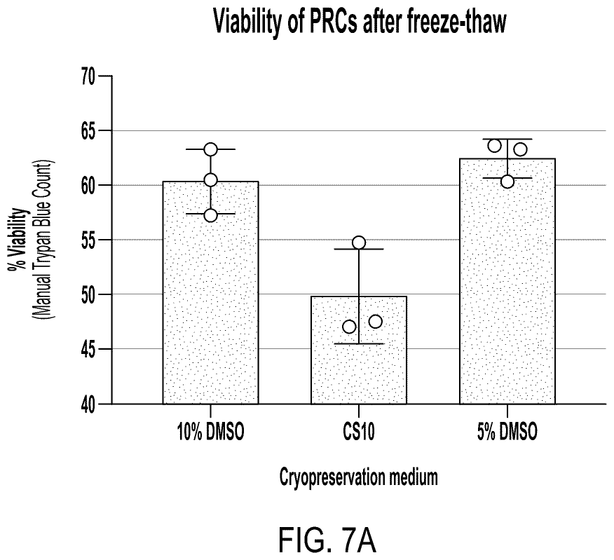

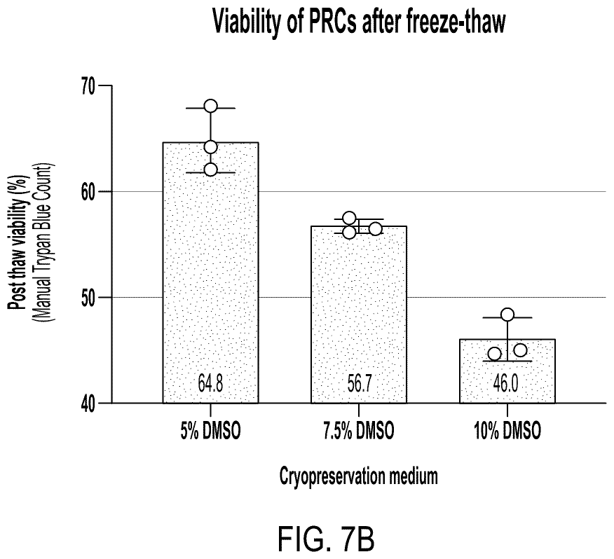

Post-thaw cell viability was evaluated using the Trypan Blue cell counting method. The formulation of the presently disclosed subject matter containing 5% DMSO, 2.5% rHA, DPBS with Ca/Mg, and 0.6% glucose showed similar viability with the formulation of the presently disclosed subject matter containing 10% DMSO, 2.5% rHA, DPBS with Ca/Mg, and 0.6% glucose (see FIG. 7A) (n=3). Both formulations of the presently disclosed subject matter showed higher viability than the commercially-available CryoStor® CS10. The viability of PRCs after freeze-thaw was also determined using formulations of the presently disclosed subject matter containing 5% DMSO, 7.5% DMSO and 10% DMSO (see FIG. 7B)

### Example 7—Applicability to Different Types of Cells (2)

Human corneal endothelial cells (CEC) derived from human induced pluripotent stem (iPS) cells were centrifuged to remove media components and any dissociation enzyme, followed by washing with 0.5% rHA/DPBS by centrifugation at 200× g for 5 minutes.

The CEC cell pellet was then resuspended with formulations comprising 2.5% rHA, 0.09% Glucose, DPBS with Ca and Mg and either 5% DMSO or 10% DMSO. Target cell density was 30,000 cells/μL.

Fifty (50) μL of formulated CEC cells were added to cryovials and cryopreserved at a freezing speed of approximately −1° C./min in Corning CoolCell® containers (Corning Life Sciences, Tewksbury, MA). After overnight storage at −80° C., vials were transferred and stored in the vapor phase of liquid nitrogen.

Post-thaw cell viability was evaluated using the trypan blue cell counting method. Vials were thawed in a 37° C. water bath and diluted with DPBS before mixing with Trypan Blue reagent. Cell viabilities were 89.3% and 88.2% for DMSO 5% and 10%-containing freezing buffer, respectively.

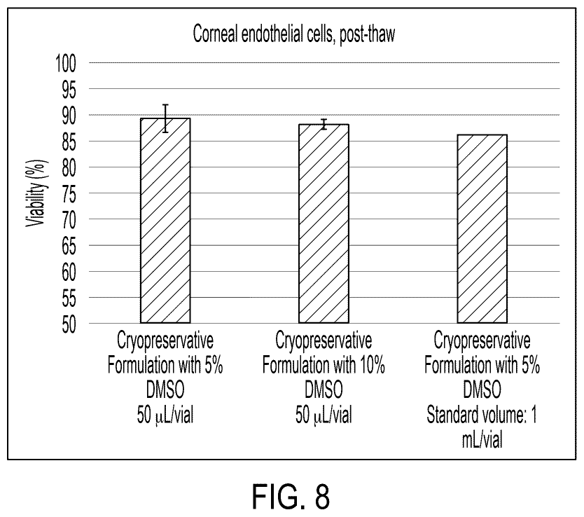

As a comparison, CEC cells were also cryopreserved at a lower cell density (3 million cells/mL) and in a standard volume (1 mL/vial) in 2.5% rHA, 0.09% glucose, DPBS with Ca and Mg, and 5% DMSO. Post-thaw cell viability under these conditions was 86.2% when determined by the Trypan Blue cell counting method (see FIG. 8).

### Example 8—Additional Candidates of the Cell Drug Product Formulation

RPE cells derived from human embryonic stem cells were pooled in a 50 mL tube and washed with 30 mL of 0.5% rHA/DPBS by centrifugation at 160× g for 5 minutes. Washing was repeated 3 times in total.

RPE cells were resuspended with 2.5% rHA/0.09%Glucose/DPBS, followed by mixing with cryoprotectant-containing buffer at a volume ratio of 1:1. Formulations containing 5% Glycerin, 200 mM Taurine or 100 mM Trehalose, 0.09% Glucose and 2.5% rHA in DPBS were prepared (Cryopreservative formulation #5 and Cryopreservative formulation #6) as shown in Table 9.

TABLE 9

| Cryopreservative | | | | | Propylene | Dextran | | | |
|---|---|---|---|---|---|---|---|---|---|
| Formulation | DMSO | rhAlbumin | Glucose | Glycerin | Glycol | 40 | Taurine | Trehalose | Buffer |
| #1 | 5 v/v % | 2.5 w/v % | 0.09 w/v % | — | — | — | — | — | DPBS |
| #2 | — | 2.5 w/v % | 0.09 w/v % | — | — | 7.5 w/v % | — | — | |
| #3 | — | 2.5 w/v % | 0.09 w/v % | 5 v/v % | — | 7.5 w/v % | — | — | |
| #4 | — | 2.5 w/v % | 0.09 w/v % | — | 5 v/v % | 7.5 w/v % | — | — | |
| #5 | — | 2.5 w/v % | 0.09 w/v % | 5 v/v % | — | — | 200 mM | — | |
| #6 | — | 2.5 w/v % | 0.09 w/v % | 5 v/v % | — | — | — | 100 mM | |

Ten (10) μL of formulated RPE cells were added to cryovials and cryopreserved using the Corning CoolCell® container (Corning Life Sciences, Tewksbury, MA) for slow cryopreservation. Vials were then transferred and stored in the vapor phase of liquid nitrogen.

Post-thaw cell viability was evaluated using the Trypan Blue cell counting method. Post-thaw cell growth was also evaluated using the CyQUANT® Cell Proliferation Assay (Thermo Fisher Scientific, Waltham, MA). Cryopreserved RPE vials were thawed, seeded and cultured in growth medium in 96 well plates. Plates were frozen down at day 2 and day 3 to evaluate DNA concentration as a cell growth index. No significant difference was observed among Cryopreservative formulation #1, Cryopreservative formulation #5 and Cryopreservative formulation #6 and no multinucleated cells which had more than four nuclei in a cell were observed among several hundred RPE cells with Cryopreservative formulation #1, Cryopreservative formulation #5, and Cryopreservative formulation #6 as shown in Table 10.

TABLE 10

| | | Normalized | Normalized | |
|---|---|---|---|---|
| | | Cell | Cell | Multi- |
| | Viability | Growth | Growth | nucleated |
| Formulation | (%) | at day 2 | at day 3 | Cells |
| Positive | 97.1 | 106.3 | 100.2 | Negative |
| Control | | | | |
| (Cryopreservative | | | | |
| Formulation #1) | | | | |
| 5% Glycerin, | 96.8 | 102.2 | 101.2 | Negative |
| 0.2M Taurine | | | | |
| (Cryopreservative | | | | |
| Formulation #5) | | | | |
| 5% Glycerin, | 98.0 | 101.7 | 107.4 | Negative |
| 0.1M Trehalose | | | | |
| (Cryopreservative | | | | |
| Formulation #6) | | | | |

Cryopreservative formulation #2 does not contain any cell penetrating cryoprotectant and thus could potentially cause toxicity to cells, and also showed high viscosity due to Dextran 40. Furthermore, multinucleated cells were observed in RPE cells cryopreserved in Cryopreservative formulation #2. While Cryopreservative formulation #3 and Cryopreservative formulation #4 showed fewer multinucleated cells, they also showed high viscosity due to Dextran 40 in the formulation. Cryopreservative formulation #5 and Cryopreservative formulation #6 lacked multinucleated cells and exhibited low viscosity due to the lack of polymeric excipients.

### Equivalents and Scope, Incorporation by Reference

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” include the singular and the plural reference unless the context clearly indicates otherwise. Thus, for example, a reference to “an agent” includes a single agent and a plurality of such agents.

Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the presently disclosed subject matter encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the presently disclosed subject matter may be explicitly excluded from any one or more of the claims. Where ranges are given, any value within the range may explicitly be excluded from any one or more of the claims. Any embodiment, element, feature, application, or aspect of the compositions and/or methods of the presently disclosed subject matter, can be excluded from any one or more claims. For purposes of brevity, all of the embodiments in which one or more elements, features, purposes, or aspects is excluded are not set forth explicitly herein.

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

What is claimed is:

1. A formulation comprising about 4-10% (v/v) cryoprotectant, about 2-8% (w/v) albumin, about 0-1.5% (w/v) glucose, and a buffer.

2. The formulation of claim 1, wherein the cryoprotectant is selected from DMSO, glycerol, and ethylene glycol.

3. The formulation of claim 1, wherein the cryoprotectant is DMSO.

4. The formulation of any one of the foregoing claims, wherein the formulation comprises about 4-6% (v/v) cryoprotectant.

5. The formulation of any one of the foregoing claims, wherein the formulation comprises about 0.08-0.10% (w/v) glucose.

6. The formulation of any one of the foregoing claims, wherein the formulation comprises about 5% (v/v) DMSO, about 2.5% (w/v) albumin, about 0.09% (w/v) glucose, and a buffer.

7. The formulation of any one of claims 1-4, wherein the formulation comprises about 0.6% (w/v) glucose.

8. The formulation of any one of claims 1-4 and 7, wherein the formulation comprises about 5% DMSO, about 2.5% albumin, about 0.6% glucose, and a buffer.

9. The formulation of any one of the foregoing claims, wherein albumin is human albumin.

10. The formulation of any one of the foregoing claims, wherein albumin is recombinant human albumin.

11. The formulation of any one of the foregoing claims, wherein the buffer is buffered saline.

12. The formulation of any one of the foregoing claims, wherein the buffer is phosphate-buffered saline (PBS).

13. The formulation of claim 11 or 12, wherein the buffered saline comprises Ca2+ and Mg2+.

14. The formulation of claim 11 or 12, wherein the buffered saline does not comprise Ca2+ and Mg2+.

15. The formulation of any one of the foregoing claims, further comprising a cell population or a tissue.

16. The formulation of claim 15, wherein the cell population is a dissociated cell population.

17. The formulation of claim 15, wherein the cell population comprises a cell monolayer, a cell sheet, cellular clusters, cellular spheres or cellular aggregates.

18. The formulation of claim 15, wherein the cell population comprises retinal pigment epithelium (RPE) cells, photoreceptor rescue cells, photoreceptor progenitor cells, or corneal endothelial cells.

19. The formulation of any one of the foregoing claims, wherein the formulation is cryopreserved.

20. The formulation of any one of the foregoing claims, wherein the formulation does not comprise a polymeric excipient.

21. The formulation of any one of the foregoing claims, wherein the formulation does not comprise dextran.

22. A cell preparation comprising
(a) the formulation of any one of claims 1-14 and
(b) a population of cells.

23. The cell preparation of claim 22, wherein the population of cells comprises retinal pigment epithelium (RPE) cells, photoreceptor rescue cells, photoreceptor progenitor cells or corneal endothelial cells.

24. The cell preparation of claim 22 or 23, wherein the population of cells is at a concentration of about 10,000 cells-100,000 cells/μL in the cell preparation.

25. The cell preparation of claim 22 or 23, wherein the population of cells is at a concentration of about 15,000-50,000 cells/μL in the cell preparation.

26. The cell preparation of any one of claims 22-25, in a cryopreservation vial, syringe, ampoule, bottle or cooler package.

27. The cell preparation of claim 26, wherein about 10 to about 500 μL of the preparation are in the vial, syringe, ampoule, bottle or cooler package.

28. The cell preparation of claim 26 or 27, wherein about 10⁵ to 10⁷ cells are present in the vial, syringe, ampoule, bottle or cooler package.

29. The cell preparation of claim 24 or 25, wherein about 10⁵ to 5×10⁶ cells are present in the vial, syringe, ampoule, bottle or cooler package.

30. The cell preparation of claim 24 or 25, wherein about 10⁵ to 2×10⁶ cells are present in the vial, syringe, ampoule, bottle or cooler package.

31. The cell preparation of any one of claims 22-30, wherein the cell preparation is cryopreserved.

32. A method of preparing a cell preparation, comprising contacting a formulation of any one of claims 1-14 with a population of cells.

33. The method of claim 32, further comprising cryopreserving the cell preparation.

34. The method of claim 32 or 33, wherein the population of cells comprises retinal pigment epithelium (RPE) cells, photoreceptor rescue cells, photoreceptor progenitor cells or corneal endothelial cells.

35. A method of treating a subject having a disorder or condition comprising,
thawing the cell preparation of claim 31, diluting the cell preparation with a diluent at a ratio selected from the group consisting of 1:2, 1:3, 1:4 and 1:5, and
administering the diluted cell preparation to the subject.

36. The method of claim 35, wherein the disorder or condition is selected from the group consisting of retinal detachment, retinal dysplasia, Angioid streaks, Myopic Macular Degeneration, or retinal atrophy or associated with a number of vision-altering ailments that result in photoreceptor damage and blindness, such as, for example, choroideremia, diabetic retinopathy, macular degeneration (e.g., age-related macular degeneration), retinitis pigmentosa, and Stargardt's Disease (fundus flavimaculatus).

37. The method of claim 35, wherein the diluent is GS2, GS2 Plus, BSS, BSS Plus®, a dextran and HSA solution, PBS, DMEM, MEM, or albumin.

38. A method of treating a subject having a disorder or condition comprising,
thawing the cell preparation of claim 31, diluting the cell preparation with a diluent at a ratio selected from the group consisting of 1:15, 1:16, 1:17, 1:18 and 1:19, and
administering the diluted cell preparation to the subject.

39. The method of claim 38, wherein the disorder or condition is selected from the group consisting of retinal detachment, retinal dysplasia, Angioid streaks, Myopic Macular Degeneration, or retinal atrophy or associated with a number of vision-altering ailments that result in photoreceptor damage and blindness, such as, for example, choroideremia, diabetic retinopathy, macular degeneration (e.g., age-related macular degeneration), retinitis pigmentosa, and Stargardt's Disease (fundus flavimaculatus).

40. The method of claim 38, wherein the diluent is GS2 or GS2 Plus, BSS, BSS Plus®, a dextran and HSA solution, PBS, DMEM, MEM, or albumin.

41. A kit comprising
(a) the cryopreserved cell preparation of claim 31,
(b) a diluent, and
(c) instructions for diluting the cell preparation prior to administration to a subject.

42. The kit of claim 41, wherein the instructions further comprise instructions for thawing the cryopreserved cell preparation.

43. A pharmaceutical composition comprising the formulation of any one of claims 1-14 in combination with a population of cells.

44. The pharmaceutical composition of claim 43, wherein the population of cells comprises retinal pigment epithelium (RPE) cells, photoreceptor rescue cells, photoreceptor progenitor cells or corneal endothelial cells.

45. The pharmaceutical composition of claim 43, wherein the population of cells comprises neural cells, neural stem cells, or neural precursor cells.

46. A pharmaceutical composition comprising the formulation of any one of claims 15-21 and a diluent, wherein the formulation to diluent ratio is in the range of 1:2 to 1:20, wherein the pharmaceutical composition is suitable for administration to a subject, optionally administration to the eye, further optionally administration to the subretina.

47. A pharmaceutical composition comprising the cell preparation of any one of claims 22-25 and 31 and a diluent, wherein the cell preparation to diluent ratio is in the range of 1:2 to 1:20, wherein the pharmaceutical composition is suitable for administration to a subject, optionally administration to the eye, further optionally administration to the subretina.

48. A cell preparation comprising the formulation of any one of claims 1-14, 19, 20 and 21 and a diluent, wherein the formulation to diluent ratio is in the range of 1:2 to 1:20, wherein the formulation comprises a cell population, wherein the cell preparation is suitable for administration to a subject, optionally administration to the eye, further optionally administration to the subretina.

49. The pharmaceutical composition of claim 46 or 47 or the cell preparation of claim 48, wherein the pharmaceutical composition or the cell preparation is not washed prior to or after dilution with the diluent.

50. The pharmaceutical composition of claim 46 or 47 or the cell preparation of claim 48, wherein the pharmaceutical composition or the cell preparation is not centrifuged prior to or after dilution with the diluent.

51. The pharmaceutical composition of claim 46 or 47 or the cell preparation of claim 48, wherein the pharmaceutical composition or the cell preparation is cryopreserved prior to dilution with the diluent.

52. The pharmaceutical composition or the cell preparation of any one of claims 48-51, wherein DMSO is present in detectable amounts at a level that is equal to or less than about 1% (v/v).

53. The pharmaceutical composition or the cell preparation of any one of claims 48-51, wherein DMSO is present in detectable amounts at a level that is equal to or less than about 0.037 mg/dose.

54. A cell preparation comprising the formulation of any one of claims 1-14, 19, 20 and 21 and a population of cells, wherein the cell preparation is thawed by dilution with a diluent and is unwashed.

55. The cell preparation of claim 54, wherein the cell preparation is diluted with a diluent at a ratio selected from the group consisting of 1:2 to 1:20.

56. The cell preparation of claim 54 or 55, wherein DMSO is present in detectable amounts at a level that is equal to or less than about 0.037 mg/dose.

57. The cell preparation of any one of claims 54-56, wherein the diluent is GS2 or GS2 Plus.
